(19) United States
(12) Patent Application Publication
(10) Pub. No.: US 2020/0163984 A1
(43) Pub. Date: May 28, 2020

(54) USE OF ISOVALERYL SPIRAMYCIN I, II AND/OR III IN MANUFACTURING MEDICAMENT FOR TREATING AND/OR PREVENTING TUMOR, AND MEDICAMENT

(71) Applicant: SHENYANG FUYANG PHARMACEUTICAL TECHNOLOGY CO., LTD., Shenyang, Liaoning (CN)

(72) Inventors: Enhong JIANG, Shenyang, Liaoning (CN); Weiqing HE, Shenyang, Liaoning (CN); Jianlu DAI, Shenyang, Liaoning (CN); Yang JIANG, Shenyang, Liaoning (CN); Xunlei JIANG, Shenyang, Liaoning (CN); Xiaofeng ZHAO, Shenyang, Liaoning (CN)

(73) Assignee: SHENYANG FUYANG PHARMACEUTICAL TECHNOLOGY CO., LTD., Shenyang, Liaoning (CN)

(21) Appl. No.: 16/627,102

(22) PCT Filed: Jul. 4, 2018
(86) PCT No.: PCT/CN2018/094504

§ 371 Date: Dec. 27, 2019

(30) Foreign Application Priority Data
Jul. 4, 2017 (CN) 201710537958.8
Dec. 27, 2017 (CN) 201711450300.X
Dec. 27, 2017 (CN) 201711450396.X
Dec. 27, 2017 (CN) 201711497546.2

(51) Int. Cl. A61K 31/7048; A61K 45/06; A61P 35/00

(52) U.S. Cl. CPC A61K 31/7048; A61P 35/00; A61K 45/06

(57) ABSTRACT

Disclosed are a medicament comprising isovaleryl spiramycin I, II and/or III, and use of isovaleryl spiramycin I, II and/or III in manufacturing medicament for treating and/or preventing tumor and the medicament.

### TECHNICAL FIELD

The present disclosure belongs to the technical field of pharmaceuticals, and specifically relates to use of isovaleryl spiramycin I, II and/or III in manufacturing medicament for the treating and/or preventing tumor and the medicament.

### BACKGROUND

Tumor is a common and frequently-occurring disease, and refers to the neoformation or neoplasm formed by clonal abnormal proliferation and differentiation caused by genetic mutation and loss of normal regulation of growth and differentiation of the histocyte of the organism under the long-term action of tumorigenic factors in vivo and in vitro. Tumors are classified into benign tumors and malignant tumors. The malignant tumors are further divided into three types: carcinomas derived from epithelial tissues, sarcomas derived from mesenchymal tissues, and carcinosarcomas. The term “cancer” is generally used to refer to all malignant tumors.

The malignant tumors are one of the major malignant diseases threatening human health and the first cause of death of the world's population. According to the latest statistics, in 2007, about 7.9 million people in the world died of various cancers, accounting for 13% of all deaths, and more than 12 million cancer cases were diagnosed, wherein 72% or more of tumor patients and deaths have occurred in underdeveloped countries, and it is rising continuously. In 2015, 9 million people in the world died of tumors, and it is expected that more than 12 million people will die of tumors in 2030. At present, the annual number of cancer cases in China is about 2.8 million, and the number of cancer deaths is more than 400,000, ranking first among all kinds of diseases in China, and showing a rising trend. With the speeding up of the pace of social life, the increasing pressure of competition, and the changes of human lifestyle and environment, tumor cases and deaths are rising year by year, and tumors have become the common diseases and the high incidence in modern society, not only seriously affecting the patients' life quality, but also bringing heavy economic and mental burden to the patients' families and the society. And tumors are also important social problems in the world, the treatment and prevention of cancer have always been one of the most pressing issues in the world. At present, chemotherapy is a main means of fighting against tumors. Although the chemotherapy has a better curative effect, the chemotherapy often causes side effects such as myelosuppression and low immune functions, making it difficult for patients to adhere to treatment. And drug resistance in the treatment process of chemotherapy has become one of the difficult problems in the current clinical treatment. In recent years, the global market of anti-tumor drugs has been growing rapidly. According to the statistics of the US FDA, the total sales of anti-cancer drugs in the world increased from 24 billion US dollars in 2004 to 39.6 billion US dollars in 2007. Although new anti-tumor drugs come out every year in the world, so far, there is still no effective means for humans to fight against cancer. At the same time, new types of cancer are constantly discovered, and the emergence and enhancement of tumor resistance/drug resistance makes the need to find new effective anti-cancer drugs more and more urgent.

Carrimycin is a new type of antibiotic with the 4″-isovaleryl spiramycin as a main component, and carrimycin is formed by cloning the 4″-o-acyl-transferase of the carbomycin producing strain into a spiramycin producing strain by a transgenic technology, directionally acylating spiramycin 4″-OH, and adding an isovaleryl side chain at the 4″-position.

Carrimycin is composed of a variety of spiramycin derivatives with the main active component isovaleryl spiramycin (I+II+III) having a total content of no less than 60%, and is a pharmaceutically acceptable pharmaceutical composition. The central structure of the main component of the carrimycin is a 16-membered lactone ring, and the 16-membered lactone ring links one molecule of forosamine, one molecule of mycaminose and one molecule of mycarose. Its main component isovaleryl spiramycin I, II, III differs from the spiramycin structure in that the group connected to the 4′-position of mycarose is isovaleryl rather than hydroxyl. The drug is jointly declared by Tonglian Shengyang Group as the 1.1 type of new drug.

The chemical structure of the main component of carrimycin is shown as in a formula (I):

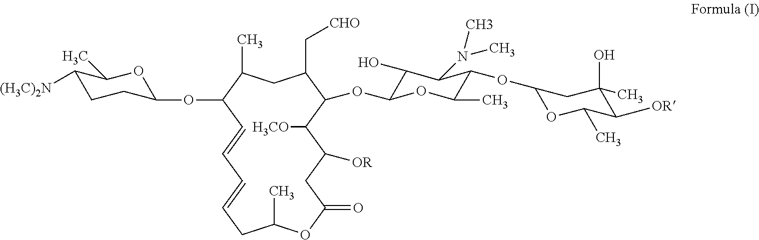

Wherein, when R═H, R′═COCH₂CH(CH₃)₂, the main component is isovaleryl spiromycin I;

When R═COCH₃, R′═COCH₂CH(CH₃)₂, the main component is isovaleryl spiromycin II;

When R═COCH₂CH₃, R′═COCH₂CH(CH₃)₂, the main component is isovaleryl spiromycin III;

Carrimycin belongs to 16-membered macrolide antibiotics, has active groups such as a carboxyl group, an alkoxy group, an epoxy group, a ketone group and an aldehyde group, and a pair of conjugated C═C, and has a molecular weight of about 884 to 982. Carrimycin and macrolide antibiotics have many commonalities due to their similar chemical structures: they are easily soluble in most organic solvents such as esters, acetone, chloroform, alcohols, etc., slightly soluble in petroleum ether, and insoluble in water. Due to the presence of two dimethylamine groups in the molecular structure, carrimycin is alkalescence and easily soluble in an acidic aqueous solution. Carrimycin has a “negative solubility” property in which the solubility decreases with an increase temperature. Since isovaleryl spiramycin, the main component of carrimycin, has a longer carbon chain at the 4″-position and a poor hydrophilicity, the solubility of carrimycin in water is smaller than that of spiramycin and 4″-acetylspiramycin.

Carrimycin is a white amorphous powder with slight hygroscopicity, and specific rotation of about −80.8°, maximum ultraviolet absorption wavelength of 231-232 nm. Carrimycin contains weak fluorescent chromophores, presents a purple reaction producing a strong purple fluorescence in case of concentrated sulfuric acid or hydrochloric acid, and has a maximum absorbance at 231-232 nm.

The drug has good lipophilicity, strong tissue penetration ability, rapid oral absorption, long body maintenance time, and sustained post antibiotic effects. According to a relationship between pharmacodynamics and chemical conformation, after the 4″-position of the macrolide antibiotics acylation, macrolide antibiotics have improved lipophilicity and in vivo activity, and significantly improved in vivo antibacterial activity and clinical therapeutic effects, and the in vivo stability of antibiotics enhances with the growing of the carbon chain of the 4″-hydroxy ester, that is, isovaleryl spiramycin>butyryl spiramycin>propionyl spiramycin>acetyl spiramycin.

The preliminary in vitro and in vivo pharmacodynamic tests showed that the drug not only has good antibacterial activity against most G⁺ bacteria, but also has certain effects on some G⁻ bacteria, and its technical indicators are obviously superior to those of azithromycin, erythromycin, acetyl spiramycin, and medemycin. It has the strongest antimicrobial activity especially against Mycoplasma pneumoniae, also has certain antimicrobial activity against the erythromycin resistant bacteria, gonococcus, pneumococcus, Staphylococcus aureus, Bacillus pyocyaneus, Bacillus influenzae, Haemophilus influenzae, Bacteroides fragilis, legionella, multi-line Bacillus and Clostridium perfringens, and a tiny cross resistance against Staphylococcus aureus with clinical resistance to the erythromycin. Carrimycin will be primarily used to treat gram-positive infections, especially upper respiratory tract infections, and may be used for urinary tract infections.

In a recent study, the applicant found that through the evaluation of isovaleryl spiramycin I, II and/or III against the in vitro antiproliferative activity of human breast cancer cells MCF-7 and MDA-MB-231, human hepatoma cells HepG2 or murine hepatoma cells H₂₂, human non-small cell lung cancer cells A549, Lewis lung cancer cells, human large cell lung cancer cells H460 and H1299, human renal clear cell adenocarcinoma cell 786-0, human renal cell adenocarcinoma cell 769-P, human glioma cell U251, human glioblastoma cell A172, human tissue lymphoma cell U937, human cervical cancer cell HeLa, human prostate cancer cell PC3, human pancreatic cancer cell PANC-1, human esophageal cancer cell TE-1, human gastric adenocarcinoma cell SGC7901, human colon cancer cell HT-29, human promyelocytic leukemia cell HL-60, human thyroid cancer cell TPC-1, and human bladder cancer cell T-24, the samples showed good antiproliferative activity against the cells tested, indicating that isovaleryl spiramycin I, II and/or III is expected to be a new drug for treating tumors, thereby completing the present disclosure.

### SUMMARY

The technical problem to be solved by the present disclosure is to overcome the defects of the prior art and provide use of isovaleryl spiramycin I, II and/or III in manufacturing medicament for treating and/or preventing tumor.

To solve the above technical problem, the present disclosure adopts the following technical solution:

The present disclosure firstly provides use of isovaleryl spiramycin I, II and/or III in manufacturing medicament for the treating and/or preventing tumor.

The tumor includes solid tumor and non-solid tumor.

Further, the solid tumor includes benign solid tumor and malignant solid tumor; the non-solid tumor is lymphoma or leukemia.

Further, the malignant solid tumor is breast cancer, liver cancer, lung cancer, renal cancer, brain tumor, cervical cancer, prostate cancer, lymphoma, pancreatic cancer, esophageal cancer, gastric cancer, colon cancer, thyroid cancer, bladder cancer, or malignant skin tumor;

Preferably, the brain tumor is glioma or meningioma, and the gastric cancer is gastric adenocarcinoma.

The present disclosure shows by experiments that isovaleryl spiramycin I, II and/or III show good antiproliferative activity on human breast cancer cells MCF-7 and MDA-MB-231, human hepatoma cells HepG2, human non-small cell lung cancer cells A549, human large cell lung cancer cells H460 and H1299, human renal clear cell adenocarcinoma cell 786-0, human renal cell adenocarcinoma cell 769-P, human tissue lymphoma cell U937, human cervical cancer cell HeLa, human prostate cancer cell PC3, human glioma cell U251, human glioblastoma cell A172, human pancreatic cancer cell PANC-1, human colon cancer cell HT-29, human esophageal cancer cell TE-1, human gastric adenocarcinoma cell SGC7901, human promyelocytic leukemia cell HL-60, human thyroid cancer cell TPC-1, and human bladder cancer cell T-24, confirming that isovaleryl spiramycin I, II and/or III can be used for treating these tumors or cancer diseases.

Further, the medicament is in various formulations made of isovaleryl spiramycin I, II and/or III and pharmaceutically acceptable salts of isovaleryl spiramycin I, II and/or III and pharmaceutically acceptable adjuvants.

Further, the medicament is in various formulations made of isovaleryl spiramycin I, II and/or III and the pharmaceutically acceptable salts of isovaleryl spiramycin I, II and/or III, and anti-tumor drugs and the pharmaceutically acceptable adjuvants.

In the present disclosure, the isovaleryl spiramycin I, II and/or III and at least one of the anti-tumor drugs can be formulated into a compound preparation.

Further, when preparing the compound preparation, the dosage ratio of isovaleryl spiramycin I, II and/or III to a second active component is 1-99: 99-1, preferably 5-95:95-5, more preferably 10-90:90-10, further preferably 20-80:80-20.

Further, the medicament is a combination of a first agent and a second agent, the first agent contains isovaleryl spiramycin I, II and/or III and the pharmaceutically acceptable salts of isovaleryl spiramycin I, II and/or III, and the second agent contains an anti-tumor drug.

In the present disclosure, the first agent containing the active component isovaleryl spiramycin I, II and/or III can be used together with the second agent containing one or more than one drug selected from a group containing a chemotherapy drug, a radiotherapy drug, a targeted therapy drug or an immunotherapeutic drug. When they are used in combination, the first agent and the second agent can be administered in no particular order, namely, the first agent may be used first, or the second agent may be used first, or both are used simultaneously.

When they are used in combination, the dosage ratio of the first agent to the second agent is 1-99:99-1, preferably 5-95:95-5, more preferably 10-90:90-10, further preferably 20-80:80-20.

Further, the anti-tumor drug is a chemotherapy drug, a radiotherapy drug, a targeted therapy drug, and/or an immunotherapeutic drug.

The present disclosure also provides a medicament for treating and/or preventing tumor, the active component of the medicament includes isovaleryl spiramycin I, II and/or III.

Further, the medicament also includes a second active component.

Further, the second active component includes one or more than one drug selected from a group containing a chemotherapy drug, a radiotherapy drug, a targeted therapy drug and an immunotherapeutic drug.

The present disclosure also provides a combination product for treating and/or preventing tumor, the combination product includes a first agent and a second agent, and the active component of the first agent is isovaleryl spiramycin I, II and/or III, and the second agent includes one or more than one drug selected from a group containing a chemotherapy drug, a radiotherapy drug, a targeted therapy drug and an immunotherapeutic drug.

Further, the medicament or the first agent is a pharmaceutically acceptable formulation.

Further, a dose of isovaleryl spiramycin I, II and/or III in the medicament or the first agent is in a range from 5 to 1,500 mg; preferably in a range from 50 to 1,000 mg; more preferably in a range from 100 to 400 mg.

In the present disclosure, isovaleryl spiramycin I can be separated and prepared according to the methods of the prior art, such as the isovaleryl spiramycin I can be separated and prepared according to a method of Example 1 in CN101785778A. Isovaleryl spiramycin II can be separated and prepared according to the methods of the prior art, such as the isovaleryl spiramycin II can be separated and prepared according to a method of Example 1 in CN101785779A. Isovaleryl spiramycin III can be separated and prepared according to the methods of the prior art, such as the isovaleryl spiramycin III can be separated and prepared according to a method of Example 1 in CN101773510A.

After adopting the above technical solution, the disclosure has the following beneficial effects compared with the prior art:

The present disclosure shows that isovaleryl spiramycin I, II and/or III have good anti-tumor effects, especially have good curative effects on tumors including breast cancer, liver cancer, lung cancer, lymphoma, cervical cancer, prostate cancer, colon cancer or leukemia. The present disclosure not only provides a theoretical basis for the application and clinical promotion of isovaleryl spiramycin I, II and/or III in the preparation of drugs for treating and/or preventing tumor, but also has important economic and social benefits.

### DETAILED DESCRIPTION OF THE EMBODIMENTS

To make the objectives, technical solutions and advantages of the embodiments of the present disclosure clearer, a clear and complete description of the technical solutions in the present embodiments will be given below. The following embodiments are used to illustrate the present disclosure, but are not used to limit the protection scope of the present disclosure.

### Embodiment 1 Isovaleryl Spiramycin I Tablets

Specification: 200 mg/350 mg

Tablet Core Prescription:

| | | |
|---|---|---|
| Isovaleryl spiramycin I | 200 | g |
| Microcrystalline cellulose | 15 | g |
| Sodium carboxymethyl starch | 22 | g |
| Povidone K₃₀ (5%) | 15 | g |
| Magnesium stearate | 3 | g |
| Prepared into | 1,000 | tablets |

Coating Liquid Prescription:

| Opadry II | 21 | g |
|---|---|---|
| Distilled water | appropriate amount | |
| Prepared into | 105 | ml |

Preparation Process:

Preparation of the tablet core: the main drug and adjuvants respectively were made to pass through a 100-mesh sieve, and a prescription amount of isovaleryl spiramycin I, a prescription amount of microcrystalline cellulose and a ½ prescription amount of sodium carboxymethyl starch were uniformly mixed, and then a 5% povidone K30 aqueous solution was added to make a soft material. A 18-mesh sieve was used for granulating, and the wet granules were dried under a ventilated condition at 60° C. for 2 hours. After the wet granules were, a 18-mesh sieve was used for dispersing the granules, and then a ½ prescription amount of sodium carboxymethyl starch and prescription amount of magnesium stearate were added. And after the materials were uniformly mixed, the mixture was tabletted with a shallow concave stamping die having a diameter of 11 mm to obtain a tablet core containing drugs, wherein the tablet is 350 mg in weight and 6.5 kg in hardness.

Preparation of the coating liquid: the required Opadry II (white) was weighed, the required amount of water was added into a liquid preparation container, the Opadry II was added into the liquid preparation container in batch. After all the Opadry II was added, the stirring speed was reduced to make the spiral disappear, and then stirring was continued to be performed for 30 min to obtain the coating liquid.

Preparation of thin film coated tablets: the tablet core was placed in a coating pan, the coating conditions were determined, and coating was carried out with the host speed of 20 r/min, the inlet air temperature of 40° C., the outlet air temperature of 30° C., the spray pressure of 0.02 Mpa, and the spray slurry flow rate of 1 ml/min. And after a constant state was achieved, the coating was continuously to be sprayed for 1.5 hours until the surfaces of the tablets were smooth and uniform in color, wherein tablets which were in compliance with the inspection standard of film coatings were qualified. The coating gains about 5% in weight.

### Embodiment 2 Isovaleryl Spiramycin I Tablets (Calculated by 10,000 Tablets)

Prescription:

| Isovaleryl spiramycin I raw powder | 1000 | g |
|---|---|---|
| Low-substituted hydroxypropyl cellulose (5%) | 92.5 | g |
| Sodium carboxymethyl starch (3%) | 55.5 | g |
| Magnesium stearate (1%) | 18.5 | g |
| Starch | Total weight- minus | |
| | the weight of other raw | |
| | and auxiliary materials | |
| Total weight | 1,850 | g |

Preparation process: an appropriate amount of starch was weighed, diluted to a concentration of 15%, and heated to a paste to get a binder; the main material isovaleryl spiramycin I, and the adjuvants starch, low-substituted hydroxypropyl cellulose, sodium carboxymethyl starch, and magnesium stearate pass through a 100-mesh sieve, respectively, and the required main material and adjuvants were weighed according to the prescription amount. After the isovaleryl spiramycin I, starch and low-substituted hydroxypropyl cellulose were fully and uniformly mixed, the starch paste with a starch concentration of 15% was used to prepare the mixture into a soft material which was granulated by a 14-mesh sieve, and granules were dried at 50-60° C. to control a water content to be 3-5%. A 14-mesh sieve was used for dispersing the granules, and then sodium carboxymethyl starch and magnesium stearate were added to be mixed, and the granule content was measured. The weight of the tablet was calculated according to the granule content, and the mixture was tabletted (with a 19 mm shallow concave punch), then the difference in the weight of the tablets was detected. After passing the test, the tablets were packaged.

### Embodiment 3 Isovaleryl Spiramycin I Capsules (Calculated by 10,000 Granules)

Prescription:

| Isovaleryl spiramycin I raw powder | 1,000 | g |
|---|---|---|
| Starch | 1,080 g minus the | |
| | weight of isovaleryl | |
| | spiramycin I raw powder | |
| No. 3 medicinal capsule | 1,000 | granules |
| Liquid paraffin | 50 | ml |

Preparation process: the main material isovaleryl spiramycin I and the adjuvant medicinal starch were separately weighed according to the process formula amount, and then fully mixed in a mixer for 1.5-2 hours. The data obtained by sampling and content testing should be basically consistent with the theoretical data (the weight of each capsule was about 0.105 g), and the qualified No. 3 medicinal capsule and the mixed raw materials to be loaded were filled in a filling device according to the operation requirements of an automatic capsule machine, and the filled capsules were subjected to a difference test (±10% or less, <0.3 g) to see if the dissolution rate meets the requirements or not, the capsules that meet the requirements after being tested were put into a polishing machine to be polished for 15-20 minutes with the liquid paraffin added, and then were taken out to be tested by finished product packaging boxes.

### Embodiment 4 Isovaleryl Spiramycin I Dry Syrup (Calculated According to 10,000 Bags)

Prescription:

| | | |
|---|---|---|
| Isovaleryl spiramycin I raw powder | 1,250 | g |
| Citric acid (0.5%) | 15 | g |

| | |
|---|---|
| Sucrose | total weight minus other |
| | raw and auxiliary materials |
| Total weight | about 500 g |
| Pigment (curcumin) | about 1 g |

Preparation process: the isovaleryl spiramycin I raw powder, citric acid and sucrose were respectively grinded into granules by a high-speed jet mill, and 85% of the granules pass through a 300-mesh sieve, 15% of the granules pass through a 180-mesh sieve. Then the fine powder after grinding was weighed according to the prescription amount and fully mixed for 1-1.5 hours, the content was measured, the loading capacity was calculated (the theoretical loading capacity is 500 mg per bag). Then the mixture was put into a bagging machine, aluminum foil paper was installed, and filling was carried out according to the operation requirements of a filling machine. The difference was allowed to be within ±5%, and after the filling, the outer packaging was carried out after passing the inspection.

### Embodiment 5 Isovaleryl Spiramycin I Granules (Calculated According to 10,000 Bags)

Prescription:

| | | |
|---|---|---|
| Isovaleryl spiramycin I raw powder | 1,250 | g |
| Powdered sugar | 20,000 | g |
| Dextrin | 9,000 | g |

| | |
|---|---|
| 5% PVP-K₃₀ | appropriate amount |

Preparation process: the isovaleryl spiramycin I raw powder, powdered sugar and dextrin were made to pass through a 120-mesh sieve, and the isovaleryl spiramycin I, powdered sugar and dextrin were weighed according to the prescription amount and uniformly mixed. And the above uniformly mixed materials were made into a soft material with a 5% PVP-K₃₀ mucilage, and then the soft material was granulated with a swinging granulation machine, dried at 70° C. and subjected to granule dispersion, and the resulting granules were subpackaged after being qualified for inspection.

### Embodiment 6 Isovaleryl Spiramycin I Freeze-Dried Powder Injection

500 mg of isovaleryl spiramycin I raw powder was uniformly mixed with an equimolar amount of adipic acid, and the mixture was dissolved in 5 ml of water to obtain a faint yellow clear solution having a pH between 4.6 and 5.6. Further, 40 mg of mannitol was added as a lyophilized proppant into the faint yellow clear solution, and after being frozen rapidly at a low temperature for 9 hours, the material was freeze-dried to obtain a faint yellow loose mass, which was dissolved in 10 ml of sterile water before being used.

### Embodiment 7 Isovaleryl Spiramycin II Tablets

Specification: 200 mg/350 mg

Tablet Core Prescription:

| | | |
|---|---|---|
| Isovaleryl spiramycin II | 200 | g |
| Microcrystalline cellulose | 110 | g |
| Sodium carboxymethyl starch | 22 | g |
| Povidone K₃₀ (5%) | 15 | g |
| Magnesium stearate | 3 | g |
| Prepared into | 1,000 | tablets |

Coating Liquid Prescription:

| Opadry II | 21 | g |
|---|---|---|
| Distilled water | appropriate amount | |
| Prepared into | 105 | ml |

Preparation Process:

Preparation of the tablet core: the main drug and adjuvants respectively were made to pass through a 100-mesh sieve, and a prescription amount of isovaleryl spiramycin II, a prescription amount of microcrystalline cellulose and a ½ prescription amount of sodium carboxymethyl starch were uniformly mixed, and then a 5% povidone K30 aqueous solution was added to make a soft material. A 18-mesh sieve was used for granulating, and the wet granules were dried under a ventilated condition at 60° C. for 2 hours. After the wet granules were dried, a 18-mesh sieve was used for dispersing the granules, and then a ½ prescription amount of sodium carboxymethyl starch and prescription amount of magnesium stearate were added. And after the materials were uniformly mixed, the mixture was tabletted with a shallow concave stamping die having a diameter of 11 mm to obtain a tablet core containing drugs, wherein the tablet is 350 mg in weight and 6.5 kg in hardness.

Preparation of the coating liquid: the required Opadry II (white) was weighed, the required amount of water was added into a liquid preparation container, the Opadry II was added into the liquid preparation container in batch. After all the Opadry II was added, the stirring speed was reduced to make the spiral disappear, and then stirring was continued to be performed for 30 min to obtain the coating liquid.

Preparation of thin film coated tablets: the tablet core was placed in a coating pan, the coating conditions were determined, and coating was carried out with the host speed of 20 r/min, the inlet air temperature of 40° C., the outlet air temperature of 30° C., the spray pressure of 0.02 Mpa, and the spray slurry flow rate of 1 ml/min. And after a constant state was achieved, the coating was continuously to be sprayed for 1.5 hours until the surfaces of the tablets were smooth and uniform in color, wherein tablets which were in compliance with the inspection standard of film coatings were qualified. The coating gains about 5% in weight.

### Embodiment 8 Isovaleryl Spiramycin II Tablets (Calculated by 10,000 Tablets)

Prescription:

| Isovaleryl spiramycin II raw powder | 1000 | g |
|---|---|---|
| Low-substituted hydroxypropyl cellulose (5%) | 92.5 | g |
| Sodium carboxymethyl starch (3%) | 55.5 | g |
| Magnesium stearate (1%) | 18.5 | g |
| Starch | Total weight minus | |
| | the weight of other raw | |
| | and auxiliary materials | |
| Total weight | 1,850 | g |

Preparation process: an appropriate amount of starch was weighed, diluted to a concentration of 15%, and heated to a paste to make a binder. The main material isovaleryl spiramycin II, and the adjuvants starch, low-substituted hydroxypropyl cellulose, sodium carboxymethyl starch, and magnesium stearate were made to pass through a 100-mesh sieve, respectively, and the required main material and adjuvants were weighed according to the prescription amount. After the isovaleryl spiramycin II, starch and low-substituted hydroxypropyl cellulose were fully and uniformly mixed, the starch paste with a starch concentration of 15% was used to prepare the mixture into a soft material which was granulated by a 14-mesh sieve, and granules were dried at 50-60° C. to control a water content to be 3-5%. A 14-mesh sieve was used for dispersing the granules, and then sodium carboxymethyl starch and magnesium stearate were added to be mixed, and the granule content was measured. The weight of the tablet was calculated according to the granule content, and the mixture was tabletted (with a 19 mm shallow concave punch), then the difference in the weight of the tablets was detected. After passing the test, the tablets were packaged.

### Embodiment 9 Isovaleryl Spiramycin II Capsules (Calculated by 10,000 Granules)

Prescription:

| Isovaleryl spiramycin II raw powder | 1,000 | g |
|---|---|---|
| Starch | 1,080 g minus the | |
| | weight of isovaleryl | |
| | spiramycin II raw powder | |
| No. 3 medicinal capsule | 1,000 | granules |
| Liquid paraffin | 50 | ml |

Preparation process: the main material isovaleryl spiramycin II and the adjuvant medicinal starch were separately weighed according to the process formula amount, and then fully mixed in a mixer for 1.5-2 hours. The data obtained by sampling and content testing should be basically consistent with the theoretical data (the weight of each capsule was about 0.105 g), and the qualified No. 3 medicinal capsule and the mixed raw materials to be loaded were filled in a filling device according to the operation requirements of an automatic capsule machine, and the filled capsules were subjected to a difference test (±10% or less, <0.3 g) to see if the dissolution rate meets the requirements or not, the capsules that meet the requirements after being tested were put into a polishing machine to be polished for 15-20 minutes with the liquid paraffin added, and then were taken out to be tested by finished product packaging boxes.

### Embodiment 10 Isovaleryl Spiramycin II Dry Syrup (Calculated According to 10,000 Bags)

Prescription:

| | | |
|---|---|---|
| Isovaleryl spiramycin II raw powder | 1,250 | g |
| Citric acid (0.5%) | 15 | g |

| | |
|---|---|
| Sucrose | total weight minus other |
| | raw and auxiliary materials |
| Total weight | about 500 g |
| Pigment (curcumin) | about 1 g |

Preparation process: the isovaleryl spiramycin II raw powder, citric acid and sucrose were respectively grinded into granules by a high-speed jet mill, and 85% of the granules pass through a 300-mesh sieve, 15% of the granules pass through a 180-mesh sieve. Then the fine powder after grinding was weighed according to the prescription amount and fully mixed for 1-1.5 hours, the content was measured, the loading capacity was calculated (the theoretical loading capacity was 500 mg per bag). Then the mixture was put into a bagging machine, aluminum foil paper was installed, and filling was carried out according to the operation requirements of a filling machine. The difference was allowed to be within ±5%, and after the filling, the outer packaging was carried out after passing the inspection.

### Embodiment 11 Isovaleryl Spiramycin II Granules (Calculated According to 10,000 Bags)

Prescription:

| | | |
|---|---|---|
| Isovaleryl spiramycin II raw powder | 1,250 | g |
| Powdered sugar | 20,000 | g |
| Dextrin | 9,000 | g |

| | |
|---|---|
| 5% PVP-K₃₀ | appropriate amount |

Preparation process: the isovaleryl spiramycin II raw powder, powdered sugar and dextrin were made to pass through a 120-mesh sieve, and the isovaleryl spiramycin II, powdered sugar and dextrin were weighed according to the prescription amount and uniformly mixed. The above uniformly mixed materials were made into a soft material with a 5% PVP-K30 mucilage, and then the soft material was granulated with a swinging granulation machine, dried at 70° C. and subjected to granule dispersion, and the resulting granules were subpackaged after being qualified for inspection.

### Embodiment 12 Isovaleryl Spiramycin II Freeze-Dried Powder Injection

500 mg of isovaleryl spiramycin II raw powder was weighed and uniformly mixed with an equimolar amount of adipic acid, and the mixture was dissolved in 5 ml of water to obtain a faint yellow clear solution having a pH between 4.6 and 5.6. Further, 40 mg of mannitol was added as a lyophilized proppant into the faint yellow clear solution, and after being frozen rapidly at a low temperature for 9 hours, the material was freeze-dried to obtain a faint yellow loose mass, which was dissolved in 10 ml of sterile water before being used.

### Embodiment 13 Isovaleryl Spiramycin III Tablets

Specification: 200 mg/350 mg

Tablet Core Prescription:

| | | |
|---|---|---|
| Isovaleryl spiramycin III | 200 | g |
| Microcrystalline cellulose | 110 | g |
| Sodium carboxymethyl starch | 22 | g |
| Povidone K₃₀ (5%) | 15 | g |
| Magnesium stearate | 3 | g |
| Prepared into | 1,000 | tablets |

Coating Liquid Prescription:

| Opadry II | 21 | g |
|---|---|---|
| Distilled water | appropriate amount | |
| Prepared into | 105 | ml |

Preparation Process:

Preparation of the tablet core: the main drug and adjuvants respectively were made to pass through a 100-mesh sieve, and a prescription amount of isovaleryl spiramycin III, a prescription amount of microcrystalline cellulose and a ½ prescription amount of sodium carboxymethyl starch were uniformly mixed, and then a 5% povidone K30 aqueous solution was added to make a soft material. A 18-mesh sieve was used for granulating, and the wet granules were dried under a ventilated condition at 60° C. for 2 hours. After the wet granules were dried, a 18-mesh sieve was used for dispersing the granules, and then a ½ prescription amount of sodium carboxymethyl starch and prescription amount of magnesium stearate were added. And after the materials were uniformly mixed, the mixture was tabletted compressed with a shallow concave stamping die having a diameter of 11 mm to obtain a tablet core containing drugs, wherein the tablet is 350 mg in weight and 6.5 kg in hardness.

Preparation of the coating liquid: the required Opadry II (white) was weighed, the required amount of water was added into a liquid preparation container, the Opadry II was added into the liquid preparation container in batch. After all the Opadry II was added, the stirring speed was reduced to make the spiral disappear, and then stirring was continued to be performed for 30 min to obtain the coating liquid.

Preparation of thin film coated tablets: the tablet core was placed in a coating pan, the coating conditions were determined, and coating was carried out with the host speed of 20 r/min, the inlet air temperature of 40° C., the outlet air temperature of 30° C., the spray pressure of 0.02 Mpa, and the spray slurry flow rate of lml/min. And after a constant state was achieved, the coating was continuously to be sprayed for 1.5 hours until the surfaces of the tablets were smooth and uniform in color, wherein tablets which were in compliance with the inspection standard of film coatings were qualified. The coating gains about 5% in weight.

### Embodiment 14 Isovaleryl Spiramycin III Tablets (Calculated by 10,000 Tablets)

Prescription:

| Isovaleryl spiramycin III raw powder | 1000 | g |
|---|---|---|
| Low-substituted hydroxypropyl | 92.5 | g |
| cellulose (5%) | | |
| Sodium carboxymethyl starch (3%) | 55.5 | g |
| Magnesium stearate (1%) | 18.5 | g |
| Starch | Total weight minus | |
| | the weight of other raw | |
| | and auxiliary materials | |
| Total weight | 1,850 | g |

Preparation process: an appropriate amount of starch was weighed, diluted to a concentration of 15%, and heated to a paste to get a binder. The main material isovaleryl spiramycin III, and the adjuvants starch, low-substituted hydroxypropyl cellulose, sodium carboxymethyl starch, and magnesium stearate were made to pass through a 100-mesh sieve, respectively, and the required main material and adjuvants were weighed according to the prescription amount. After the isovaleryl spiramycin III, starch and low-substituted hydroxypropyl cellulose were fully and uniformly mixed, the starch paste with a starch concentration of 15% was used to prepare the mixture into a soft material which was granulated by a 14-mesh sieve, and granules were dried at 50-60° C. to control a water content to be 3-5%. A 14-mesh sieve was used for dispersing the granules, and then sodium carboxymethyl starch and magnesium stearate were added to be mixed, and the granule content was measured. The weight of the tablet was calculated according to the granule content, and the mixture was tabletted (with a Φ9 mm shallow concave punch), then the difference in the weight of the tablets was detected. After passing the test, the tablets were packaged.

### Embodiment 15 Isovaleryl Spiramycin III Capsules (Calculated by 10,000 Granules)

Prescription:

| Isovaleryl spiramycin III raw powder | 1,000 | g |
|---|---|---|
| Starch | 1,080 g minus the | |
| | weight of isovaleryl | |
| | spiramycin III raw powder | |
| No. 3 medicinal capsule | 1,000 | granules |
| Liquid paraffin | 50 | ml |

Preparation process: the main material isovaleryl spiramycin III and the adjuvant medicinal starch were separately weighed according to the process formula amount, and then fully mixed in a mixer for 1.5-2 hours. The data obtained by sampling and content testing should be basically consistent with the theoretical data (the weight of each capsule was about 0.105 g), and the qualified No. 3 medicinal capsule and the mixed raw materials to be loaded were filled in a filling device according to the operation requirements of an automatic capsule machine, and the filled capsules were subjected to a difference test (±10% or less, <0.3 g) to see if the dissolution rate meets the requirements or not, the capsules that meet the requirements after being tested were put into a polishing machine to be polished for 15-20 minutes with the liquid paraffin added, and then were taken out to be tested by finished product packaging boxes.

### Embodiment 16 Isovaleryl Spiramycin III Dry Syrup (Calculated According to 10,000 Bags)

Prescription

| Isovaleryl spiramycin III raw powder | 1,250 | g |
|---|---|---|
| Citric acid (0.5%) | 15 | g |
| Sucrose | total weight minus other | |
| | raw and auxiliary materials | |
| Total weight | about 500 | g |
| Pigment (curcumin) | about 1 | g |

Preparation process: the isovaleryl spiramycin III raw powder, citric acid and sucrose were respectively grinded into granules by a high-speed jet mill, and 85% of the granules pass through a 300-mesh sieve, 15% of the granules pass through a 180-mesh sieve. And then the fine powder after grinding was weighed according to the prescription amount and fully mixed for 1-1.5 hours, the content was measured, the loading capacity was calculated (the theoretical loading capacity was 500 mg per bag). Then the mixture was put into a bagging machine, aluminum foil paper was installed, and filling was carried out according to the operation requirements of a filling machine. The difference was allowed to be within ±5%, and after the filling, the outer packaging was carried out after passing the inspection.

### Embodiment 17 Isovaleryl Spiramycin III Granules (Calculated According to 10,000 Bags)

Prescription:

| | | |
|---|---|---|
| Isovaleryl spiramycin III raw powder | 1,250 | g |
| Powdered sugar | 20,000 | g |
| Dextrin | 9,000 | g |

| | |
|---|---|
| 5% PVP-K₃₀ | appropriate amount |

Preparation process: the isovaleryl spiramycin III raw powder, powdered sugar and dextrin were made to pass through a 120-mesh sieve, and the isovaleryl spiramycin III, powdered sugar and dextrin were weighed according to the prescription amount and uniformly mixed. The above uniformly mixed materials were made into a soft material with a 5% PVP-K₃₀ mucilage, and then the soft material was granulated with a swinging granulation machine, dried at 70° C. and subjected to granule dispersion, and the resulting granules were subpackaged after being qualified for inspection.

### Embodiment 18 Isovaleryl Spiramycin III Freeze-Dried Powder Injection

500 mg of isovaleryl spiramycin III raw powder was uniformly mixed with an equimolar amount of adipic acid, and the mixture was dissolved in 5 ml of water to obtain a faint yellow clear solution having a pH between 4.6 and 5.6. Further, 40 mg of mannitol was added as a lyophilized proppant into the faint yellow clear solution, and after being frozen rapidly at a low temperature for 9 hours, the material was freeze-dried to obtain a faint yellow loose mass, which was dissolved in 10 ml of sterile water before being used.

### Test Example 1 Bioassay of Anti-Tumor Activity

The purpose of the assay is to evaluate the in vitro cell proliferation inhibition or cytotoxic activity of a tested sample.

Cell Strains:

Human breast cancer cells MCF-7 and MDA-MB-231, human hepatoma cells HepG2, human non-small cell lung cancer cells A549, human large cell lung cancer cells H460 and H1299, human renal clear cell adenocarcinoma cell 786-0, human renal cell adenocarcinoma cell 769-P, human glioma cell U251, human glioblastoma cell A172, human tissue lymphoma cell U937, human cervical cancer cell HeLa, human prostate cancer cell PC3, human pancreatic cancer cell PANC-1, human esophageal cancer cell TE-1, human gastric adenocarcinoma cell SGC7901, human colon cancer cell HT-29, human promyelocytic leukemia cell HL-60, human thyroid cancer cell TPC-1, and human bladder cancer cell T-24.

Reagents:

RPMI1640 medium, MEM medium, DMEM low sugar medium, fetal calf serum purchased from Gibco, USA, trypsin, glutamine, penicillin, streptomycin, dimethyl sulfoxide (DMSO), and methyl-thiazol-tetrazolium (MTT) purchased from Sigma, USA.

Instruments:

Carbon dioxide incubator (Sanyo, Japan), enzyme-linked immunosorbent analyzer (Tecan, Austria), 96-well culture plate (Corning, USA), inverted microscope (Motic, China).

The operation steps are as follows:

Adherent Cells:

MCF-7, MDA-MB-231, HepG2, A549, H460, H1299, 786-0, 769-P, U251, A172, HeLa, PC3, PANC-1, TE-1, SGC7901, and HT-29 were adherent tumor cells. The adherent tumor cells in the logarithmic growth phase were selected and digested with trypsin, then were prepared into a 4 to 5×10⁴/ml cell suspension by a medium containing 10% fetal bovine serum. And the cell suspension was inoculated in a 96-well culture plate, and each well was 100 μL. The 96-well culture plate was cultured at 37° C. and 5% CO₂ for 24 hours. The experimental group was replaced with a new culture medium containing different concentrations of the sample to be tested, namely, isovaleryl spiramycin I, isovaleryl spiramycin II, or isovaleryl spiramycin III, while the control group was replaced with a culture medium containing the same volume of solvent. Each group was set up with 3 parallel wells that were cultured at 37° C. and 5% CO₂ for 48 hours. After the supernatant was removed, the wells were washed carefully for 3 times with PBS. And 100 μL of freshly prepared culture medium containing 0.5 mg/ml MTT was added to each well for continuous incubation for 4 hours at 37° C. After the supernatant was removed carefully, 150 μL of DMSO was added to each well, and after the material was mixed for 10 minutes with a micro-oscillator, the optical density value was measured at 492 nm with a microplate reader.

Suspension Cells:

U937 and HL-60 were suspension cells, and cells in a logarithmic growth phase were selected and prepared into a 2×10⁵/ml cell suspension by a RPMI 1640 culture medium containing 10% fetal bovine serum. And the cell suspension was inoculated in a 96-well culture plate, and each well was 50 μL and the 96-well culture plate was cultured at 37° C. and 5% CO₂ for 24 hours. 50 μL of a culture medium containing different concentrations of the tested sample isovaleryl spiramycin I, isovaleryl spiramycin II, or isovaleryl spiramycin III was added in the experimental group, while a culture medium containing the same volume of solvent was added into the control group. Each group was set up with 3 parallel wells that were cultured at 37° C. and 5% CO₂ for 48 h. And 10 μL of freshly prepared medium containing 5 mg/ml MTT was added into each well for continuous incubation for 4 hours at 37° C. The crystals were dissolved in 100 μL of a triple solution (SDS 10 g, 10 M HCl 0.1 mL, isobutanol 5 mL, diluted with distilled water to 100 mL), and incubated at 37° C. for 12 hours. The optical density value was measured at 492 nm with a microplate reader.

Evaluation of Results:

The inhibition rate of the medicament on tumor cell growth is calculated according to the following formula:

Tumor cell growth inhibition rate (%)=[A492 (negative control)−A492 (dosing group)]/A492 (negative control)×100%

And the half-inhibitory concentration (IC₅₀) of the sample is determined therefrom.

Results:

The evaluation results of in vitro antiproliferative activity of the samples selected from human breast cancer cells MCF-7 and MDA-MB-231, human hepatoma cells HepG2, human non-small cell lung cancer cells A549, human large cell lung cancer cells H460 and H1299, human renal clear cell adenocarcinoma cell 786-O, human renal cell adenocarcinoma cell 769-P, human glioma cell U251, human glioblastoma cell A172, human tissue lymphoma cell U937, human cervical cancer cell HeLa, human prostate cancer cell PC3, human pancreatic cancer cell PANC-1, human esophageal cancer cell TE-1, human gastric adenocarcinoma cell SGC7901, human colon cancer cell HT-29, human promyelocytic leukemia cell HL-60, human thyroid cancer cell TPC-1, and human bladder cancer cell T-24 are shown in Table 1, Table 2 and Table 3:

TABLE 1
Inhibition of isovaleryl spiramycin
I on the proliferation of tumor cells

| | IC₅₀ | | IC₅₀ |
|---|---|---|---|
| Cell Strain | (μg/mL) | Cell Strain | (μg/mL) |
| MCF-7 | 20.79 ± 1.57 | A172 | 10.24 ± 0.37 |
| MDA-MB-231 | 18.12 ± 0.61 | U937 | 10.88 ± 0.05 |
| HepG2 | 17.90 ± 1.74 | HeLa | 10.31 ± 0.27 |
| A549 | 19.93 ± 1.66 | PC3 | 9.39 ± 0.85 |
| H460 | 19.31 ± 0.35 | PANC-1 | 9.96 ± 0.46 |
| H1299 | 24.03 ± 2.07 | TE-1 | 8.42 ± 1.53 |
| 786-O | 5.08 ± 0.08 | SGC-7901 | 11.28 ± 1.27 |
| 769-P | 5.09 ± 0.04 | HT-29 | 17.30 ± 0.52 |
| U251 | 11.01 ± 0.32 | HL-60 | 16.52 ± 1.26 |
| TPC-1 | 19.97 ± 1.93 | T-24 | 18.68 ± 0.58 |

TABLE 2
Inhibition of isovaleryl spiramycin II
on the proliferation of tumor cells

| | IC₅₀ | | IC₅₀ |
|---|---|---|---|
| Cell Strain | (μg/mL) | Cell Strain | (μg/mL) |
| MCF-7 | 51.14 ± 2.54 | A172 | 33.62 ± 0.57 |
| MDA-MB-231 | 49.60 ± 0.39 | U937 | 34.92 ± 0.81 |
| HepG2 | 37.94 ± 1.71 | HeLa | 33.31 ± 0.40 |
| A549 | 36.97 ± 2.92 | PC3 | 32.88 ± 0.53 |
| H460 | 41.16 ± 0.71 | PANC-1 | 31.93 ± 0.12 |
| H1299 | 42.24 ± 0.44 | TE-1 | 35.59 ± 2.64 |
| 786-O | 20.18 ± 0.86 | SGC-7901 | 39.14 ± 1.21 |
| 769-P | 20.61 ± 0.48 | HT-29 | 31.05 ± 3.23 |
| U251 | 35.35 ± 1.57 | HL-60 | 29.39 ± 1.82 |
| TPC-1 | 45.37 ± 2.90 | T-24 | 39.85 ± 3.26 |

TABLE 3
Inhibition of isovaleryl spiramycin III
on the proliferation of tumor cells

| | IC₅₀ | | IC₅₀ |
|---|---|---|---|
| Cell Strain | (μg/mL) | Cell Strain | (μg/mL) |
| MCF-7 | 32.44 ± 1.18 | A172 | 16.17 ± 0.54 |
| MDA-MB-231 | 29.47 ± 0.28 | U937 | 15.59 ± 0.08 |
| HepG2 | 27.42 ± 3.52 | HeLa | 15.86 ± 0.62 |
| A549 | 28.84 ± 1.73 | PC3 | 16.37 ± 0.27 |
| H460 | 28.80 ± 0.19 | PANC-1 | 13.18 ± 0.23 |
| H1299 | 32.40 ± 0.41 | TE-1 | 20.19 ± 1.83 |
| 786-O | 10.52 ± 0.56 | SGC-7901 | 17.26 ± 0.79 |
| 769-P | 10.75 ± 0.56 | HT-29 | 22.68 ± 2.32 |
| U251 | 16.46 ± 1.54 | HL-60 | 21.38 ± 1.75 |
| TPC-1 | 29.72 ± 4.02 | T-24 | 31.23 ± 1.47 |

The available results show that the samples isovaleryl spiramycin I, isovaleryl spiramycin II, and isovaleryl spiramycin III show good anti-proliferative activity against the cells tested.

### Test Example 2 In Vivo Test

1. Inhibition of Isovaleryl Spiramycin I, II and III on Human Large Cell Lung Cancer Cells 11460 in Nude Mice Model

Establishment of a Mouse Solid Tumor Model

H460 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 4, and Table 5).

The tumor growth inhibition rates of isovaleryl spiramycin I groups with low, medium and high doses are 20.70%, 46.33% and 70.11%, respectively.

The tumor growth inhibition rates of isovaleryl spiramycin II groups with low, medium and high doses are 14.22%, 34.43% and 61.12%, respectively.

The tumor growth inhibition rates of isovaleryl spiramycin III groups with low, medium and high doses are 17.41%, 23.31% and 63.93%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 56.56%, 49.00% and 31.96% respectively.

The relative tumor proliferation rates of isovaleryl spiramycin II groups with low, medium and high doses are 51.97%, 46.49% and 37.89% respectively.

The relative tumor proliferation rates of isovaleryl spiramycin III groups with low, medium and high doses are 63.03%, 42.54% and 35.95% respectively.

There are no significant changes in the mouse weight of isovaleryl spiramycin I, II and III groups with low, medium and high doses compared with the model group.

TABLE 4
Effect of isovaleryl spiramycin I, II and III on the inhibition rate of transplanted
tumor of human large cell lung cancer H460 cells in nude mice (x̅ ± s)

| | | Number of | Body | Body | | |
|---|---|---|---|---|---|---|
| | Dose | Animals | Weight (g) | Weight (g) | Tumor | Inhibition |
| Group | (mg/kg) | (Start/End) | (d 1) | (d 30) | Weight (g) | Rate (%) |
| Model Group | 0 | 6/6 | 20.47 ± 0.41 | 24.53 ± 1.74 | 1.72 ± 0.18 | — |
| Cyclophosphamide | 30 | 6/6 | 20.46 ± 0.54 | 21.20 ± 0.75 | 0.50 ± 0.06*** | 71.08 |
| Isovaleryl | 25 | 6/6 | 20.67 ± 0.33 | 22.55 ± 1.90 | 1.37 ± 0.13** | 20.70 |
| spiramycin I | 50 | 6/6 | 20.45 ± 0.62 | 23.54 ± 1.18 | 0.93 ± 0.18*** | 46.33 |
| | 100 | 6/6 | 20.58 ± 0.32 | 23.91 ± 1.34 | 0.52 ± 0.07*** | 70.11 |
| Isovaleryl | 25 | 6/6 | 20.72 ± 0.47 | 24.10 ± 1.02 | 1.48 ± 0.24 | 14.22 |
| spiramycin II | 50 | 6/6 | 20.58 ± 0.83 | 22.49 ± 2.46 | 1.13 ± 0.08** | 34.43 |
| | 100 | 6/6 | 20.61 ± 0.69 | 24.87 ± 0.74 | 0.67 ± 0.20*** | 61.12 |
| Isovaleryl | 25 | 6/6 | 20.39 ± 0.56 | 25.09 ± 1.38 | 1.42 ± 0.26* | 17.41 |
| spiramycin III | 50 | 6/6 | 20.56 ± 0.47 | 24.94 ± 0.68 | 1.32 ± 0.25* | 23.31 |
| | 100 | 6/6 | 20.56 ± 0.64 | 25.07 ± 1.48 | 0.62 ± 0.26*** | 63.93 |

*p < 0.05 compared with the model group,
**p < 0.01 compared with the model group,
***p < 0.001 compared with the model group

TABLE 5
Effect of isovaleryl spiramycin I, II and III on the volume change of transplanted
tumor of human large cell lung cancer H460 cells in nude mice (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3) (d 30) | Volume (RTV) | Rate (T/C) |
| Model Group | 0 | 6/6 | 146.29 ± 28.15 | 1497.79 ± 178.37 | 10.51 ± 1.89 | |
| Cyclophosphamide | 30 | 6/6 | 146.98 ± 27.55 | 468.49 ± 128.57*** | 3.24 ± 0.99*** | 30.86 |
| Isovaleryl | 25 | 6/6 | 148.20 ± 15.93 | 889.44 ± 343.94** | 5.94 ± 2.02** | 56.56 |
| spiramycin I | 50 | 6/6 | 148.51 ± 17.68 | 753.94 ± 306.82** | 5.15 ± 2.25** | 49.00 |
| | 100 | 6/6 | 145.08 ± 19.59 | 477.54 ± 89.21*** | 3.36 ± 0.85*** | 31.96 |
| Isovaleryl | 25 | 6/6 | 147.44 ± 15.40 | 804.17 ± 292.57 | 5.46 ± 1.87 | 51 97 |
| spiramycin II | 50 | 6/6 | 146.77 ± 18.26 | 700.40 ± 143.83** | 4.88 ± 1.36** | 46.49 |
| | 100 | 6/6 | 149.76 ± 13.12 | 604.38 ± 195.98*** | 3.98 ± 1.10*** | 37.89 |
| Isovaleryl | 25 | 6/6 | 140.44 ± 14.04 | 914.05 ± 279.14** | 6.62 ± 2.34* | 63.03 |
| spiramycin III | 50 | 6/6 | 148.59 ± 14.47 | 666.74 ± 160.61*** | 4.47 ± 0.94*** | 42.54 |
| | 100 | 6/6 | 143.19 ± 17.40 | 531.56 ± 78.33*** | 3.78 ± 0.84*** | 35.95 |

*p < 0.05 compared with the model group;
**p < 0.01 compared with the model group,
***p < 0.001 compared with the model group

2. Inhibition of Isovaleryl Spiramycin I, II and III on Human Non-Small Cell Lung Cancer Cell 111299 in Nude Mice Model

Establishment of a Mouse Solid Tumor Model

H1299 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 6, and Table 7).

The tumor growth inhibition rates of isovaleryl spiramycin I groups with low, medium and high doses are 22.11%, 43.83% and 69.95%, respectively.

The tumor growth inhibition rates of isovaleryl spiramycin II groups with low, medium and high doses are 17.32%, 44.21% and 58.37%, respectively.

The tumor growth inhibition rates of isovaleryl spiramycin III groups with low, medium and high doses are 13.11%, 49.38% and 62.78%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 89.42%, 49.81% and 27.43% respectively.

The relative tumor proliferation rates of isovaleryl spiramycin II groups with low, medium and high doses are 83.01%, 46.94% and 36.86% respectively.

The relative tumor proliferation rates of isovaleryl spiramycin III groups with low, medium and high doses are 89.88%, 48.11% and 32.43% respectively.

There are no significant changes in the mouse weight of isovaleryl spiramycin I, II and III groups with low, medium and high doses compared with the model group.

TABLE 6
Effect of isovaleryl spiramycin I, II and III on the inhibition rate of transplanted
tumor of human non-small cell lung cancer cell H1299 in nude mice (x̅ ± s)

| | | Number of | Body | Body | | |
|---|---|---|---|---|---|---|
| | Dose | Animals | Weight (g) | Weight (g) | Tumor | Inhibition |
| Group | (mg/kg) | (Start/End) | (d 1) | (d 30) | Weight (g) | Rate (%) |
| Model Group | 0 | 6/6 | 21.85 ± 0.77 | 27.73 ± 1.52 | 1.74 ± 0.21 | |
| Cyclophosphamide | 30 | 6/6 | 21.83 ± 0.33 | 23.72 ± 2.41 | 0.38 ± 0.08*** | 78.00 |
| Isovaleryl | 25 | 6/6 | 21.82 ± 0.41 | 28.49 ± 1.23 | 1.36 ± 0.33* | 22.11 |
| spiramycin I | 50 | 6/6 | 21.74 ± 1.04 | 27.99 ± 2.47 | 0.98 ± 0.27*** | 43.83 |
| | 100 | 6/6 | 21.64 ± 0.96 | 27.62 ± 2.63 | 0.52 ± 0.21*** | 69.95 |
| Isovaleryl | 25 | 6/6 | 21.91 ± 0.85 | 28.06 ± 2.64 | 1.44 ± 0.29* | 17.32 |
| spiramycin II | 50 | 6/6 | 21.99 ± 1.18 | 25.65 ± 5.03 | 0.97 ± 0.16*** | 44.21 |
| | 100 | 6/6 | 21.54 ± 0.88 | 28.81 ± 1.21 | 0.73 ± 0.25*** | 58.37 |
| Isovaleryl | 25 | 6/6 | 21.73 ± 0.32 | 28.91 ± 1.32 | 1.51 ± 0.30 | 13.11 |
| spiramycin III | 50 | 6/6 | 21.82 ± 0.53 | 27.84 ± 2.73 | 0.88 ± 0.20*** | 49.38 |
| | 100 | 6/6 | 21.77 ± 0.58 | 27.71 ± 1.48 | 0.65 ± 0.17*** | 62.78 |

*p < 0.05 compared with the model group,
**p < 0.01 compared with the model group,
***p < 0.001 compared with the model group

TABLE 7
Effect of isovaleryl spiramycin I, II and III on the volume change of transplanted
tumor of human non-small cell lung cancer cell H1299 in nude mice (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3) (d 30) | Volume (RTV) | Rate (T/C) |
| Model Group | 0 | 6/6 | 136.68 ± 18.16 | 1710 ± 163.11 | 12.82 ± 2.82 | |
| Cyclophosphamide | 30 | 6/6 | 135.46 ± 16.48 | 384.21 ± 106.99 | 2.91 ± 0.95*** | 22.72 |
| Isovaleryl | 25 | 6/6 | 136.07 ± 17.13 | 1513.14 ± 387.19* | 11.46 ± 3.83 | 89.42 |
| spiramycin I | 50 | 6/6 | 135.51 ± 20.47 | 861.71 ± 164.32*** | 6.39 ± 1.01** | 49.81 |
| | 100 | 6/6 | 137.34 ± 22.95 | 470.36 ± 21.77*** | 3.52 ± 0.68*** | 27.43 |
| Isovaleryl | 25 | 6/6 | 139.13 ± 12.26 | 1474.57 ± 104.54 | 10.64 ± 0.92 | 83.01 |
| spiramycin II | 50 | 6/6 | 138.94 ± 20.16 | 821.57 ± 90.36*** | 6.02 ± 1.07*** | 46.94 |
| | 100 | 6/6 | 136.94 ± 14.60 | 634.05 ± 180.18*** | 4.72 ± 1.57*** | 36.86 |
| Isovaleryl | 25 | 6/6 | 137.58 ± 14.95 | 1565.97 ± 277.97 | 11.52 ± 2.51 | 89.88 |
| spiramycin III | 50 | 6/6 | 137.82 ± 17.12 | 816.87 ± 299.03** | 6.17 ± 2.64*** | 48.11 |
| | 100 | 6/6 | 137.09 ± 12.95 | 557.82 ± 209.73*** | 4.16 ± 1.90*** | 32.43 |

*p < 0.05 compared with the model group;
**p < 0.01 compared with the model group,
***p < 0.001 compared with the model group

3. Inhibition of Isovaleryl Spiramycin I, II and III on Human Esophageal Cancer in Nude Mice Model

Establishment of a Mouse Solid Tumor Model

TE-1 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a cyclophosphamide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 8, and Table 9).

The tumor growth inhibition rates of isovaleryl spiramycin I groups with low, medium and high doses are 30.92%, 51.01% and 69.71%, respectively.

The tumor growth inhibition rates of isovaleryl spiramycin II groups with low, medium and high doses are 24.87%, 43.78% and 72.48%, respectively.

The tumor growth inhibition rates of isovaleryl spiramycin III groups with low, medium and high doses are 22.64%, 40.17% and 65.46%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 60.55%, 40.70% and 20.61% respectively.

The relative tumor proliferation rates of isovaleryl spiramycin II groups with low, medium and high doses are 70.32%, 50.51% and 36.49% respectively.

The relative tumor proliferation rates of isovaleryl spiramycin III groups with low, medium and high doses are 66.52%, 50.71% and 30.72% respectively.

There are no significant changes in the mouse weight of isovaleryl spiramycin I, II and III groups with low, medium and high doses compared with the model group.

TABLE 8
Effect of isovaleryl spiramycin I, II and III on the inhibition rate of transplanted
tumor of human esophageal cancer cell TE-1 in nude mice (x̅ ± s)

| | | Number of | Body | Body | | |
|---|---|---|---|---|---|---|
| | Dose | Animals | Weight (g) | Weight (g) | Tumor | Inhibition |
| Group | (mg/kg) | (Start/End) | (d 1) | (d 30) | Weight (g) | Rate (%) |
| Model Group | 0 | 6/6 | 22.67 ± 0.87 | 20.97 ± 0.97 | 1.57 ± 0.62 | — |
| Cyclophosphamide | 30 | 6/6 | 23.18 ± 1.22 | 20.57 ± 1.14 | 0.44 ± 0.18 ** | 71.87 |
| Isovaleryl | 12.5 | 6/6 | 23.27 ± 1.35 | 21.75 ± 0.60 | 1.08 ± 0.24 | 30.92 |
| spiramycin I | 25 | 6/6 | 22.98 ± 1.83 | 21.28 ± 1.04 | 0.77 ± 0.43 * | 51.01 |
| | 50 | 6/6 | 22.52 ± 1.19 | 22.32 ± 0.52 | 0.48 ± 0.36 ** | 69.71 |
| Isovaleryl | 12.5 | 6/6 | 21.43 ± 1.58 | 21.97 ± 0.99 | 1.18 ± 0.60 | 24.87 |
| spiramycin II | 25 | 6/6 | 22.93 ± 0.41 | 22.28 ± 0.66 | 0.88 ± 0.46 | 43.78 |
| | 50 | 6/6 | 22.35 ± 1.07 | 21.62 ± 0.32 | 0.43 ± 0.26 ** | 72.48 |
| Isovaleryl | 12.5 | 6/6 | 22.52 ± 0.57 | 21.38 ± 0.37 | 1.21 ± 0.53 | 22.64 |
| spiramycin III | 25 | 6/6 | 21.82 ± 1.34 | 21.38 ± 0.67 | 0.94 ± 0.39 | 40.17 |
| | 50 | 6/6 | 21.23 ± 1.05 | 20.70 ± 0.63 | 0.54 ± 0.22 ** | 65.46 |

* p < 0.05 compared with the model group,
** p < 0.01 compared with the model group,
***p < 0.001 compared with the model group

TABLE 9
Effect of isovaleryl spiramycin I, II and III on the volume change of transplanted
tumor of human esophageal cancer cell TE-1 in nude mice (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3) (d 30) | Volume (RTV) | Rate (T/C) |
| Model Group | 0 | 6/6 | 218.70 ± 76.83 | 2378.49 ± 829.69*** | 11.89 ± 15.80*** | — |
| Cyclophosphamide | 30 | 6/6 | 269.96 ± 92.18 | 886.22 ± 271.22*** | 4.00 ± 1.91*** | 30.18 |
| Isovaleryl | 12.5 | 6/6 | 408.51 ± 150.80 | 2690.27 ± 374.79*** | 5.48 ± 3.80*** | 60.55 |
| spiramycin I | 25 | 6/6 | 258.44 ± 104.69 | 1143.85 ± 402.36** | 3.94 ± 2.71** | 40.70 |
| | 50 | 6/6 | 279.56 ± 156.70 | 626.64 ± 431.35 | 3.65 ± 1.71 | 20.61 |
| Isovaleryl | 12.5 | 6/6 | 134.33 ± 57.77 | 1027.37 ± 363.55** | 7.92 ± 6.86** | 70.32 |
| spiramycin II | 25 | 6/6 | 195.76 ± 76.95 | 1075.36 ± 720.20* | 6.99 ± 5.39* | 50.51 |
| | 50 | 6/6 | 143.85 ± 16.25 | 570.92 ± 293.97* | 6.97 ± 7.85* | 36.49 |
| Isovaleryl | 12.5 | 6/6 | 173.45 ± 63.55 | 1254.78 ± 637.73** | 6.03 ± 5.27** | 66.52 |
| spiramycin III | 25 | 6/6 | 206.72 ± 79.72 | 1140.10 ± 819.08* | 5.02 ± 6.81* | 50.71 |
| | 50 | 6/6 | 331.09 ± 208.02 | 1106.23 ± 865.11 | 3.60 ± 1.29 | 30.72 |

*p < 0.05 compared with the model group;
**p < 0.01 compared with the model group,
***p < 0,001 compared with the model group

4. Inhibition of Isovaleryl Spiramycin I, II and III on Human Gastric Adenocarcinoma in Nude Mice Model

Establishment of a Mouse Solid Tumor Model

SGC7901 cells in a logarithmic growth phase were taken and subjected to a trypan blue exclusion experiment showing that the cell viability is was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a cyclophosphamide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 10, and Table 11).

The tumor growth inhibition rates of isovaleryl spiramycin I groups with low, medium and high doses are 31.56%, 53.13% and 70.78%, respectively.

The tumor growth inhibition rates of isovaleryl spiramycin II groups with low, medium and high doses are 24.44%, 41.76% and 70.24%, respectively.

The tumor growth inhibition rates of isovaleryl spiramycin III groups with low, medium and high doses are 21.68%, 41.13% and 63.34%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 61.13%, 42.67% and 20.23% respectively.

The relative tumor proliferation rates of isovaleryl spiramycin II groups with low, medium and high doses are 70.48%, 51.42% and 36.95% respectively.

The relative tumor proliferation rates of isovaleryl spiramycin III groups with low, medium and high doses are 65.48%, 49.44% and 30.34% respectively.

There are no significant changes in the mouse weight of isovaleryl spiramycin I, II and III groups with low, medium and high doses compared with the model group.

TABLE 10
Effect of isovaleryl spiramycin I, II and III on the inhibition rate of transplanted
tumor of human gastric adenocarcinoma cell SGC7901 in nude mice (x̅ ± s)

| | | Number of | Body | Body | | |
|---|---|---|---|---|---|---|
| | Dose | Animals | Weight (g) | Weight (g) | Tumor | Inhibition |
| Group | (mg/kg) | (Start/End) | (d 1) | (d 30) | Weight (g) | Rate (%) |
| Model Group | 0 | 6/6 | 22.38 ± 0.71 | 21.08 ± 0.76 | 1.97 ± 0.61 | — |
| Cyclophosphamide | 30 | 6/6 | 22.63 ± 1.35 | 19.85 ± 0.72 | 0.44 ± 0.41 *** | 72.26 |
| Isovaleryl | 12.5 | 6/6 | 22.02 ± 0.76 | 21.63 ± 0.80 | 1.07 ± 0.53 * | 31.56 |
| spiramycin I | 25 | 6/6 | 22.05 ± 1.55 | 21.12 ± 0.90 | 0.74 ± 0.52 ** | 53.13 |
| | 50 | 6/6 | 22.65 ± 0.95 | 21.87 ± 0.61 | 0.46 ± 0.36 *** | 70.78 |
| Isovaleryl | 12.5 | 6/6 | 22.45 ± 1.20 | 21.73 ± 0.98 | 1.19 ± 0.62 | 24.44 |
| spiramycin II | 25 | 6/6 | 22.65 ± 0.81 | 22.12 ± 0.85 | 0.91 ± 0.53 ** | 41.76 |
| | 50 | 6/6 | 22.13 ± 1.35 | 21.40 ± 0.38 | 0.47 ± 0.42 *** | 70.24 |
| Isovaleryl | 12.5 | 6/6 | 22.68 ± 0.45 | 21.55 ± 0.63 | 1.23 ± 0.70 | 21.68 |
| spiramycin III | 25 | 6/6 | 21.98 ± 1.37 | 21.55 ± 0.95 | 0.92 ± 0.40 ** | 41.13 |
| | 50 | 6/6 | 21.42 ± 1.10 | 20.57 ± 0.87 | 0.58 ± 0.52 ** | 63.34 |

* p < 0.05 compared with the model group,
** p < 0.01 compared with the model group,
*** p < 0.001 compared with the model group

TABLE 11
Effect of isovaleryl spiramycin I, II and III on the volume change of transplanted
tumor of human gastric adenocarcinoma cell SGC7901 in nude mice (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3)(d 30) | Volume (RTV) | Rate (T/C) |
| Model Group | 0 | 6/6 | 212.82 ± 68.03 | 2373.38 ± 834.85*** | 11.15 ± 12.27*** | — |
| Cyclophosphamide | 30 | 6/6 | 252.63 ± 74.57 | 865.27 ± 422.15* | 4.27 ± 2.36* | 30.71 |
| Isovaleryl | 12.5 | 6/6 | 412.96 ± 157.62 | 2815.50 ± 770.23*** | 5.37 ± 3.46*** | 61.13 |
| spiramycin I | 25 | 6/6 | 258.63 ± 104.26 | 1230.76 ± 635.23* | 3.98 ± 2.78* | 42.67 |
| | 50 | 6/6 | 279.56 ± 156.70 | 630.69 ± 458.86 | 3.64 ± 1.65 | 20.23 |
| Isovaleryl | 12.5 | 6/6 | 134.33 ± 57.77 | 1029.72 ± 818.01* | 7.90 ± 6.97* | 70.48 |
| spiramycin II | 25 | 6/6 | 195.76 ± 76.95 | 1094.82 ± 687.01* | 6.88 ± 5.66* | 51.42 |
| | 50 | 6/6 | 143.85 ± 16.25 | 577.98 ± 411.47* | 6.90 ± 7.37* | 36.95 |
| Isovaleryl | 12.5 | 6/6 | 186.22 ± 80.26 | 1326.06 ± 96.10** | 5.52 ± 4.03** | 65.48 |
| spiramycin III | 25 | 6/6 | 274.12 ± 83.50 | 1473.96 ± 798.75** | 3.77 ± 6.51** | 49.44 |
| | 50 | 6/6 | 295.39 ± 188.69 | 974.67 ± 839.14 | 3.66 ± 1.35 | 30.34 |

*p < 0.05 compared with the model group,
**p < 0.01 compared with the model group,
***p < 0.001 compared with the model group

5. Inhibition of Isovaleryl Spiramycin I, II and III on Human Prostate Cancer in Nude Mice Model

Establishment of a Mouse Solid Tumor Model

PC3 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a cyclophosphamide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 12, and Table 13).

The tumor growth inhibition rates of isovaleryl spiramycin I groups with low, medium and high doses are 33.87%, 51.33% and 71.01%, respectively.

The tumor growth inhibition rates of isovaleryl spiramycin II groups with low, medium and high doses are 23.49%, 40.16% and 50.44%, respectively.

The tumor growth inhibition rates of isovaleryl spiramycin III groups with low, medium and high doses are 25.44%, 40.16% and 60.37%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 70.43%, 49.14% and 30.72%, respectively.

The relative tumor proliferation rates of isovaleryl spiramycin II groups with low, medium and high doses are 83.04%, 60.08% and 44.48%, respectively.

The relative tumor proliferation rates of isovaleryl spiramycin III groups with low, medium and high doses are 75.75%, 55.02% and 34.57%, respectively.

There are no significant changes in the mouse weight of isovaleryl spiramycin I, II and III groups with low, medium and high doses compared with the model group.

TABLE 12
Effect of isovaleryl spiramycin I, II and III on the inhibition rate of transplanted
tumor of human prostate cancer cell PC3 in nude mice (x̅ ± s)

| | | Number of | Body | Body | | |
|---|---|---|---|---|---|---|
| | Dose | Animals | Weight (g) | Weight (g) | Tumor | Inhibition |
| Group | (mg/kg) | (Start/End) | (d 1) | (d 30) | Weight (g) | Rate (%) |
| Model Group | 0 | 6/6 | 21.3 ± 1.28 | 22.27 ± 1.95 | 1.88 ± 0.75 | — |
| Cyclophosphamide | 30 | 6/6 | 21.57 ± 0.75 | 16.43 ± 0.78 | 0.56 ± 0.17** | 70.03 |
| Isovaleryl | 25 | 6/6 | 21.17 ± 0.92 | 22.8 ± 0.93 | 1.24 ± 0.51 | 33.87 |
| spiramycin I | 50 | 6/6 | 21.63 ± 0.98 | 22.96 ± 0.60 | 0.91 ± 0.33* | 51.33 |
| | 100 | 6/6 | 20.92 ± 0.58 | 21.97 ± 0.85 | 0.55 ± 0.21** | 71.01 |
| Isovaleryl | 25 | 6/6 | 21.78 ± 0.58 | 21.93 ± 0.85 | 1.44 ± 0.56 | 23.49 |
| spiramycin II | 50 | 6/6 | 20.98 ± 0.8 | 21.68 ± 0.41 | 1.13 ± 0.43 | 40.16 |
| | 100 | 6/6 | 21.37 ± 0.88 | 21.52 ± 0.60 | 0.93 ± 0.27* | 50.44 |
| Isovaleryl | 25 | 6/6 | 21.3 ± 0.75 | 21.67 ± 0.73 | 1.40 ± 0.54 | 25.43 |
| spiramycin III | 50 | 6/6 | 21.3 ± 0.76 | 21.65 ± 0.94 | 1.12 ± 0.45* | 40.16 |
| | 100 | 6/6 | 21.4 ± 0.69 | 21.57 ± 0.48 | 0.75 ± 0.25** | 60.37 |

*p < 0.05 compared with the model group,
**p < 0.01 compared with the model group,
***p < 0.001 compared with the model group

TABLE 13
Effect of isovaleryl spiramycin I, II and III on the volume change of transplanted
tumor of human prostate cancer cell PC3 in nude mice (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3) (d 30) | Volume (RTV) | Rate (T/C) |
| Model Group | 0 | 6/6 | 36.77 ± 41.07 | 2458.27 ± 93.21 | 19.56 ± 0.65 | — |
| Cyclophosphamide | 30 | 6/6 | 155.36 ± 34.96 | 834.50 ± 135.35** | 5.73 ± 2.23** | 29.33 |
| Isovaleryl | 25 | 6/6 | 127.59 ± 6.73 | 1758.15 ± 412.68 | 13.78 ± 3.15 | 70.43 |
| spiramycin I | 50 | 6/6 | 186.59 ± 39.40 | 1706.14 ± 347.50 | 9.61 ± 3.15 | 49.14 |
| | 100 | 6/6 | 175.94 ± 12.32 | 1041.69 ± 247.71* | 6.01 ± 1.78** | 30.72 |
| Isovaleryl | 25 | 6/6 | 154.86 ± 4.11 | 2404.85 ± 672.33 | 16.24 ± 6.00 | 83.04 |
| spiramycin II | 50 | 6/6 | 157.25 ± 38.97 | 1729.14 ± 128.57 | 11.75 ± 3.78 | 60.08 |
| | 100 | 6/6 | 131.11 ± 22.05 | 1162.46 ± 495.46 | 8.70 ± 2.61 | 44.48 |
| Isovaleryl | 25 | 6/6 | 146.91 ± 6.70 | 1914.85 ± 729.54 | 14.82 ± 7.55 | 75.75 |
| spiramycin III | 50 | 6/6 | 187.01 ± 50.28 | 1935.79 ± 228.06 | 10.76 ± 2.24 | 55.01 |
| | 100 | 6/6 | 155.11 ± 7.98 | 1051.03 ± 272.23* | 6.76 ± 1.61** | 34.57 |

*p < 0.05 compared with the model group;
**p < 0.01 compared with the model group,
***p < 0.001 compared with the model group

6. Inhibition of Isovaleryl Spiramycin I, II and III on Human Breast Cancer in Nude Mice Model

Establishment of a Mouse Solid Tumor Model

MCF-7 cells in a logarithmic growth phase were taken and subjected to trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a cyclophosphamide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 14, and Table 15).

The tumor growth inhibition rates of isovaleryl spiramycin I groups with low, medium and high doses are 31.10%, 51.72% and 70.12%, respectively.

The tumor growth inhibition rates of isovaleryl spiramycin II groups with low, medium and high doses are 20.55%, 41.72% and 56.81%, respectively.

The tumor growth inhibition rates of isovaleryl spiramycin III groups with low, medium and high doses are 26.75%, 39.08% and 61.78%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 71.92%, 49.05% and 30.80%, respectively.

The relative tumor proliferation rates of isovaleryl spiramycin II groups with low, medium and high doses are 79.23%, 60.58% and 44.44%, respectively.

The relative tumor proliferation rates of isovaleryl spiramycin III groups with low, medium and high doses are 75.03%, 54.92% and 34.91%, respectively.

There are no significant changes in the mouse weight of isovaleryl spiramycin I, II and III groups with low, medium and high doses compared with the model group.

TABLE 14
Effect of isovaleryl spiramycin I, II and III on the inhibition rate of transplanted
tumor of human breast cancer cell MCF-7 in nude mice (x̅ ± s)

| | | Number of | Body | Body | | |
|---|---|---|---|---|---|---|
| | Dose | Animals | Weight (g) | Weight (g) | Tumor | Inhibition |
| Group | (mg/kg) | (Start/End) | (d 1) | (d 30) | Weight (g) | Rate (%) |
| Model Group | 0 | 6/6 | 21.8 ± 0.91 | 21.58 ± 0.84 | 2.72 ± 0.34 | |
| Cyclophosphamide | 30 | 6/6 | 22.03 ± 0.70 | 17.77 ± 0.65 | 0.81 ± 0.25*8 | 70.18 |
| Isovaleryl | 25 | 6/6 | 21.33 ± 0.79 | 21.73 ± 0.95 | 1.87 ± 0.25 | 31.10 |
| spiramycin I | 50 | 6/6 | 21.22 ± 0.41 | 21.48 ± 0.73 | 1.31 ± 0.50 | 51.72 |
| | 100 | 6/6 | 22.25 ± 0.92 | 21.8 ± 0.78 | 0.81 ± 0.08** | 70.12 |
| Isovaleryl | 25 | 6/6 | 21.3 ± 0.67 | 21.78 ± 0.68 | 2.16 ± 0.51 | 20.55 |
| spiramycin II | 50 | 6/6 | 21.1 ± 0.53 | 21.37 ± 0.67 | 1.58 ± 0.51 | 41.72 |
| | 100 | 6/6 | 21.93 ± 0.62 | 21.96 ± 0.68 | 1.17 ± 0.47 | 56.81 |
| Isovaleryl | 25 | 6/6 | 20.73 ± 0.97 | 21.21 ± 0.78 | 1.99 ± 0.81 | 26.75 |
| spiramycin III | 50 | 6/6 | 21.33 ± 0.84 | 21.2 ± 0.75 | 1.66 ± 0.66 | 39.09 |
| | 100 | 6/6 | 21.53 ± 0.56 | 21.73 ± 0.69 | 1.03 ± 0.54* | 61.78 |

*p < 0.05 compared with the model group,
**p < 0.01 compared with the model group,
***p < 0.001 compared with the model group

TABLE 15
Effect of isovaleryl spiramycin I, II and III on the volume change of transplanted
tumor of human breast cancer cell MCF-7 in nude mice (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3) (d 30) | Volume (RTV) | Rate (T/C) |
| Model Group | 0 | 6/6 | 137.63 ± 12.68 | 2393.99 ± 69.20 | 17.50 ± 1.47 | |
| Cyclophosphamide | 30 | 6/6 | 103.78 ± 12.65 | 532.62 ± 56.27** | 5.17 ± 0.64** | 29.56 |
| Isovaleryl | 25 | 6/6 | 139.97 ± 26.58 | 1634.85 ± 789.61 | 12.59 ± 7.49 | 71.92 |
| spiramycin I | 50 | 6/6 | 146.57 ± 32.97 | 1162.80 ± 394.71 | 8.58 ± 4.41* | 49.05 |
| | 100 | 6/6 | 124.02 ± 15.14 | 656.95 ± 49.58** | 5.39 ± 0.97** | 30.80 |
| Isovaleryl | 25 | 6/6 | 104.64 ± 10.69 | 1450.36 ± 218.20 | 13.87 ± 1.53 | 79.23 |
| spiramycin II | 50 | 6/6 | 105.81 ± 11.20 | 1113.42 ± 71.14 | 10.60 ± 1.10 | 60.58 |
| | 100 | 6/6 | 119.69 ± 14.63 | 914.69 ± 130.26* | 7.78 ± 1.71* | 44.45 |
| Isovaleryl | 25 | 6/6 | 137.99 ± 20.99 | 1758.49 ± 129.06 | 13.13 ± 3.15 | 75.03 |
| spiramycin III | 50 | 6/6 | 109.95 ± 13.42 | 1043.33 ± 113.01 | 9.61 ± 1.66 | 54.92 |
| | 100 | 6/6 | 113.14 ± 9.02 | 684.21 ± 115.00** | 5.17 ± 0.64** | 34.90 |

*p < 0.05 compared with the model group;
**p < 0.01 compared with the model group,
***p < 0.001 compared with the model group

7. Inhibition of Isovaleryl Spiramycin I, II and III on Human Breast Cancer in Nude Mice Model

Establishment of a Mouse Solid Tumor Model

MDA-MB-231 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a cyclophosphamide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 16, and Table 17).

The tumor growth inhibition rates of isovaleryl spiramycin I groups with low, medium and high doses are 22.84%, 55.17% and 69.11%, respectively.

The tumor growth inhibition rates of isovaleryl spiramycin II groups with low, medium and high doses are 18.17%, 29.66% and 58.91%, respectively.

The tumor growth inhibition rates of isovaleryl spiramycin III groups with low, medium and high doses are 24.35%, 21.01% and 62.93%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 69.71%, 44.18% and 27.74%, respectively.

The relative tumor proliferation rates of isovaleryl spiramycin II groups with low, medium and high doses are 80.22%, 58.78% and 30.89%, respectively.

The relative tumor proliferation rates of isovaleryl spiramycin III groups with low, medium and high doses were 68.36%, 50.12% and 35.27%, respectively.

There are no significant changes in the mouse weight of isovaleryl spiramycin I, II and III groups with low, medium and high doses compared with the model group.

TABLE 16
Effect of isovaleryl spiramycin I, II and III on the inhibition rate of transplanted
tumor of human breast cancer cell MDA-MB-231 in nude mice (x̅ ± s)

| | | Number of | Body | Body | | |
|---|---|---|---|---|---|---|
| | Dose | Animals | Weight (g) | Weight (g) | Tumor | Inhibition |
| Group | (mg/kg) | (Start/End) | (d 1) | (d 30) | Weight (g) | Rate (%) |
| Model Group | 0 | 6/6 | 21.25 ± 1.34 | 24.60 ± 1.71 | 2.32 ± 0.22 | — |
| Cyclophosphamide | 30 | 6/6 | 21.65 ± 1.50 | 19.26 ± 1.49 | 0.38 ± 0.27*** | 70.54 |
| Isovaleryl | 25 | 6/6 | 21.97 ± 1.25 | 24.25 ± 1.39 | 1.79 ± 0.34* | 22.84 |
| spiramycin I | 50 | 6/6 | 21.24 ± 1.40 | 25.56 ± 1.69 | 1.04 ± 0.44*** | 55.17 |
| | 100 | 6/6 | 21.87 ± 1.49 | 24.47 ± 1.85 | 0.72 ± 0.48*** | 69.11 |
| Isovaleryl | 25 | 6/6 | 20.24 ± 1.89 | 21.32 ± 1.85 | 1.90 ± 0.31* | 18.17 |
| spiramycin II | 50 | 6/6 | 20.02 ± 1.78 | 22.92 ± 1.54 | 1.63 ± 0.32** | 29.66 |
| | 100 | 6/6 | 21.91 ± 1.97 | 22.42 ± 1.41 | 0.95 ± 0.42*** | 58.91 |
| Isovaleryl | 25 | 6/6 | 21.69 ± 1.78 | 23.97 ± 1.81 | 1.76 ± 0.20** | 24.35 |
| spiramycin III | 50 | 6/6 | 21.17 ± 1.6 | 24.36 ± 1.20 | 1.11 ± 0.54** | 52.01 |
| | 100 | 6/6 | 20.43 ± 2.77 | 23.53 ± 1.58 | 0.86 ± 0.44*** | 62.93 |

*p < 0.05 compared with the model group,
**p < 0.01 compared with the model group,
***p < 0.001 compared with the model group

TABLE 17
Effect of isovaleryl spiramycin I, II and III on the volume change of transplanted
tumor of human breast cancer cell MDA-MB-231 in nude mice (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3) (d 30) | Volume (RTV) | Rate (T/C) |
| Model Group | 0 | 6/6 | 121.07 ± 9.76 | 1875.22 ± 104.10 | 15.62 ± 2.08 | — |
| Cyclophosphamide | 30 | 6/6 | 116.90 ± 7.18 | 691.84 ± 337.21*** | 5.89 ± 2.84*** | 37.73 |
| Isovaleryl | 25 | 6/6 | 111.07 ± 8.47 | 1213.38 ± 299.29** | 10.89 ± 2.52* | 69.71 |
| spiramycin I | 50 | 6/6 | 116.00 ± 8.13 | 807.45 ± 319.26*** | 6.90 ± 2.43*** | 44.18 |
| | 100 | 6/6 | 111.91 ± 10.26 | 458.55 ± 338.74*** | 4.33 ± 3.47*** | 27.74 |
| Isovaleryl | 25 | 6/6 | 119.72 ± 6.52 | 1493.96 ± 171.49** | 12.53 ± 1.78* | 80.22 |
| spiramycin II | 50 | 6/6 | 119.07 ± 3.19 | 1094.89 ± 291.37*** | 9.18 ± 2.38*** | 58.78 |
| | 100 | 6/6 | 115.89 ± 9.01 | 700.44 ± 192.38*** | 6.08 ± 1.74*** | 30.89 |
| Isovaleryl | 25 | 6/6 | 114.53 ± 6.52 | 1217.28 ± 267.06** | 10.68 ± 2.47** | 68.36 |
| spiramycin III | 50 | 6/6 | 112.00 ± 10.40 | 860.48 ± 286.44*** | 7.83 ± 2.97*** | 50.12 |
| | 100 | 6/6 | 115.76 ± 7.04 | 647.02 ± 338.62*** | 5.51 ± 2.72*** | 35.27 |

*p < 0.05 compared with the model group;
**p < 0.01 compared with the model group,
***p < 0.001 compared with the model group

8. Inhibition of Isovaleryl Spiramycin I, II and III on Human Pancreatic Cancer in Nude Mice Model

Establishment of a Mouse Solid Tumor Model

PANC-1 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a cyclophosphamide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 18, and Table 19).

The tumor growth inhibition rates of isovaleryl spiramycin I groups with low, medium and high doses are 31.10%, 51.72% and 70.12%, respectively.

The tumor growth inhibition rates of isovaleryl spiramycin II groups with low, medium and high doses are 20.55%, 41.72% and 56.81%, respectively.

The tumor growth inhibition rates of isovaleryl spiramycin III groups with low, medium and high doses are 26.75%, 39.08% and 61.78%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 71.92%, 49.05% and 30.80%, respectively.

The relative tumor proliferation rates of isovaleryl spiramycin II groups with low, medium and high doses are 79.23%, 60.58% and 44.44%, respectively.

The relative tumor proliferation rates of isovaleryl spiramycin III groups with low, medium and high doses are 75.03%, 54.92% and 34.91%, respectively.

There are no significant changes in the mouse weight of isovaleryl spiramycin I, II and III groups with low, medium and high doses compared with the model group.

TABLE 18
Effect of isovaleryl spiramycin I, II and III on the inhibition rate of transplanted
tumor of human pancreatic cancer cell PANC-1 in nude mice (x̅ ± s)

| | | Number of | Body | Body | | Inhibition |
|---|---|---|---|---|---|---|
| | Dose | Animals | Weight (g) | Weight (g) | Tumor Weight | Rate |
| Group | (mg/kg) | (Start/End) | (d1) | (d30) | (g) | (%) |
| Model | 0 | 6/6 | 21.8 ± 0.91 | 21.58 ± 0.84 | 2.72 ± 0.34 | |
| Group | | | | | | |
| Cyclophos- | 30 | 6/6 | 22.03 ± 0.70 | 17.77 ± 0.65 | 0.81 ± 0.25*8 | 70.18 |
| phamide | | | | | | |
| Isovaleryl | 12.5 | 6/6 | 21.33 ± 0.79 | 21.73 ± 0.95 | 1.87 ± 0.25 | 31.10 |
| spiramycin | 25 | 6/6 | 21.22 ± 0.41 | 21.48 ± 0.73 | 1.31 ± 0.50 | 51.72 |
| I | 50 | 6/6 | 22.25 ± 0.92 | 21.8 ± 0.78 | 0.81 ± 0.08** | 70.12 |
| Isovaleryl | 12.5 | 6/6 | 21.3 ± 0.67 | 21.78 ± 0.68 | 2.16 ± 0.51 | 20.55 |
| spiramycin | 25 | 6/6 | 21.1 ± 0.53 | 21.37 ± 0.67 | 1.58 ± 0.51 | 41.72 |
| II | 50 | 6/6 | 21.93 ± 0.62 | 21.96 ± 0.68 | 1.17 ± 0.47 | 56.81 |
| Isovaleryl | 12.5 | 6/6 | 20.73 ± 0.97 | 21.21 ± 0.78 | 1.99 ± 0.81 | 26.75 |
| spiramycin | 25 | 6/6 | 21.33 ± 0.84 | 21.2 ± 0.75 | 1.66 ± 0.66 | 39.09 |
| III | 50 | 6/6 | 21.53 ± 0.56 | 21.73 ± 0.69 | 1.03 ± 0.54* | 61.78 |

*p < 0.05 compared with the model group,
**p < 0.01 compared with the model group,
***p < 0.001 compared with the model group

TABLE 19
Effect of isovaleryl spiramycin I, II and III on the volume change of transplanted
tumor of human pancreatic cancer cell PANC-1 in nude mice (x̅ ± s)

| | | | | | | Relative |
|---|---|---|---|---|---|---|
| | | | | | Relative | Tumor |
| | | Number of | Tumor | | Tumor | Proliferation |
| | Dose | Animals | Volume | Tumor Volume | Volume | Rate |
| Group | (mg/kg) | (Start/End) | (mm3) (d1) | (mm3) (d30) | (RTV) | (T/C) |
| Model | 0 | 6/6 | 137.63 ± 12.68 | 2393.99 ± 69.20 | 17.50 ± 1.47 | |
| Group | | | | | | |
| Cyclophos- | 30 | 6/6 | 103.78 ± 12.65 | 532.62 ± 56.27** | 5.17 ± 0.64** | 29.56 |
| phamide | | | | | | |
| Isovaleryl | 12.5 | 6/6 | 139.97 ± 26.58 | 1634.85 ± 789.61 | 12.59 ± 7.49 | 71.92 |
| spiramycin | 25 | 6/6 | 146.57 ± 32.97 | 1162.80 ± 394.71** | 8.58 ± 4.41* | 49.05 |
| I | 50 | 6/6 | 124.02 ± 15.14 | 656.95 ± 49.58*** | 5.39 ± 0.97** | 30.80 |
| Isovaleryl | 12.5 | 6/6 | 104.64 ± 10.69 | 1450.36 ± 218.20 | 13.87 ± 1.53 | 79.23 |
| spiramycin | 25 | 6/6 | 105.81 ± 11.20 | 1113.42 ± 71.14 | 10.60 ± 1.10 | 60.58 |
| II | 50 | 6/6 | 119.69 ± 14.63 | 914.69 ± 130.26** | 7.78 ± 1.71* | 44.45 |
| Isovaleryl | 12.5 | 6/6 | 137.99 ± 20.99 | 1758.49 ± 129.06 | 13.13 ± 3.15 | 75.03 |
| spiramycin | 25 | 6/6 | 109.95 ± 13.42 | 1043.33 ± 113.01 | 9.61 ± 1.66 | 54.92 |
| III | 50 | 6/6 | 113.14 ± 9.02 | 684.21 ± 115.00** | 5.17 ± 0.64** | 34.90 |

*p < 0.05 compared with the model group;
**p < 0.01 compared with the model group,
***p < 0.001 compared with the model group

9. Inhibition of Isovaleryl Spiramycin I, II and III on Human Hepatoma in Nude Mice Model

Establishment of a Mouse Solid Tumor Model

HepG-2 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a cyclophosphamide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 20, and Table 21).

The tumor growth inhibition rates of isovaleryl spiramycin I groups with low, medium and high doses are 24.43%, 57.93% and 68.22%, respectively.

The tumor growth inhibition rates of isovaleryl spiramycin II groups with low, medium and high doses are 21.07%, 31.43% and 61.56%, respectively.

The tumor growth inhibition rates of isovaleryl spiramycin III groups with low, medium and high doses of are 38.92%, 60.54% and 63.28%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 65.03%, 42.12% and 27.49%, respectively.

The relative tumor proliferation rates of isovaleryl spiramycin II groups with low, medium and high doses are 81.03%, 57.02% and 39.31%, respectively.

The relative tumor proliferation rates of isovaleryl spiramycin III groups with low, medium and high doses are 64.69%, 43.18% and 32.71%, respectively.

There are no significant changes in the mouse weight of isovaleryl spiramycin I, II and III groups with low, medium and high doses compared with the model group.

TABLE 20
Effect of isovaleryl spiramycin I, II and III on the inhibition rate of transplanted
tumor of human hepatoma cell HepG-2 in nude mice (x̅ ± s)

| | | Number of | Body | Body | | Inhibition |
|---|---|---|---|---|---|---|
| | Dose | Animals | Weight (g) | Weight (g) | Tumor Weight | Rate |
| Group | (mg/kg) | (Start/End) | (d1) | (d30) | (g) | (%) |
| Model | 0 | 6/6 | 21.21 ± 1.42 | 23.75 ± 1.71 | 2.43 ± 0.23 | — |
| Group | | | | | | |
| Cyclophos- | 30 | 6/6 | 21.89 ± 1.84 | 19.67 ± 1.49 | 0.07 ± 0.38*** | 71.04 |
| phamide | | | | | | |
| Isovaleryl | 25 | 6/6 | 21.36 ± 1.67 | 23.24 ± 1.33 | 1.84 ± 0.23** | 24.43 |
| spiramycin | 50 | 6/6 | 21.47 ± 1.58 | 23.64 ± 1.67 | 1.02 ± 0.45*** | 57.93 |
| I | 100 | 6/6 | 21.37 ± 1.36 | 22.867 ± 1.57 | 0.77 ± 0.44*** | 68.22 |
| Isovaleryl | 25 | 6/6 | 21.98 ± 1.74 | 23.35 ± 1.83 | 1.92 ± 0.32* | 21.07 |
| spiramycin | 50 | 6/6 | 20.75 ± 1.86 | 22.22 ± 1.62 | 1.67 ± 0.32** | 31.43 |
| II | 100 | 6/6 | 21.48 ± 1.98 | 22.45 ± 1.28 | 0.93 ± 0.41*** | 61.56 |
| Isovaleryl | 25 | 6/6 | 21.07 ± 1.24 | 23.94 ± 1.67 | 1.48 ± 0.42** | 38.92 |
| spiramycin | 50 | 6/6 | 21.25 ± 1.86 | 24.36 ± 1.53 | 0.96 ± 0.44*** | 60.54 |
| III | 100 | 6/6 | 20.47 ± 2.24 | 23.64 ± 1.44 | 0.89 ± 0.46*** | 63.28 |

*p < 0.05 compared with the model group;
**p < 0.01 compared with the model group,
***p < 0.001 compared with the model group

TABLE 21
Effect of isovaleryl spiramycin I, II and III on the volume change of transplanted
tumor of human hepatoma cell HepG-2 n nude mice (x̅ ± s)

| | | | | | | Relative |
|---|---|---|---|---|---|---|
| | | | | | Relative | Tumor |
| | | Number of | Tumor | | Tumor | Proliferation |
| | Dose | Animals | Volume | Tumor Volume | Volume | Rate |
| Group | (mg/kg) | (Start/End) | (mm3) (d1) | (mm3) (d30) | (RTV) | (T/C) |
| Model | 0 | 6/6 | 117.98 ± 9.34 | 1960.30 ± 92.59 | 16.74 ± 2.02 | — |
| Group | | | | | | |
| Cyclophos- | 30 | 6/6 | 119.66 ± 7.65 | 792.56 ± 287.31*** | 6.58 ± 2.18*** | 39.27 |
| phamide | | | | | | |
| Isovaleryl | 25 | 6/6 | 114.02 ± 11.68 | 1248.33 ± 337.59** | 10.89 ± 2.67** | 65.03 |
| spiramycin | 50 | 6/6 | 122.31 ± 4.68 | 851.37 ± 371.80*** | 7.05 ± 3.35*** | 42.12 |
| I | 100 | 6/6 | 110.06 ± 9.58 | 481.16 ± 326.94*** | 4.60 ± 3.39*** | 27.49 |
| Isovaleryl | 25 | 6/6 | 122.82 ± 4.10 | 1666.11 ± 202.07* | 13.57 ± 1.64* | 81.03 |
| spiramycin | 50 | 6/6 | 118.60 ± 4.30 | 1127.90 ± 319.12** | 9.55 ± 2.84*** | 57.02 |
| II | 100 | 6/6 | 118.70 ± 7.20 | 783.73 ± 312.41*** | 6.58 ± 2.48*** | 39.31 |
| Isovaleryl | 25 | 6/6 | 116.67 ± 8.32 | 1261.48 ± 283.95** | 10.83 ± 2.47** | 64.69 |
| spiramycin | 50 | 6/6 | 119.43 ± 10.86 | 852.87 ± 293.29*** | 7.23 ± 2.57*** | 43.18 |
| III | 100 | 6/6 | 118.26 ± 6.57 | 641.99 ± 364.25*** | 5.48 ± 3.19*** | 32.71 |

*p < 0.05 compared with the model group,
**p < 0.01 compared with the model group,
***p < 0.001 compared with the model group

10. Inhibition of Isovaleryl Spiramycin I, II and III on Human Non-Small Cell Lung Cancer in Nude Mice Model

Establishment of a Mouse Solid Tumor Model

A549 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a cyclophosphamide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 22, and Table 23).

The tumor growth inhibition rates of isovaleryl spiramycin I groups with low, medium and high doses are 24.00%, 58.10% and 69.52%, respectively.

The tumor growth inhibition rates of isovaleryl spiramycin II groups with low, medium and high doses are 20.73%, 31.87% and 60.19%, respectively.

The tumor growth inhibition rates of isovaleryl spiramycin III groups with low, medium and high doses are 37.99%, 55.95% and 66.53%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 67.18%, 41.93% and 28.35%, respectively.

The relative tumor proliferation rates of isovaleryl spiramycin II groups with low, medium and high doses are 83.41%, 58.75% and 39.42%, respectively.

The relative tumor proliferation rates of isovaleryl spiramycin III groups with low, medium and high doses are 65.93%, 47.25% and 33.04%, respectively.

There are no significant changes in the mouse weight of isovaleryl spiramycin I, II and III groups with low, medium and high doses compared with the model group.

TABLE 22
Effect of isovaleryl spiramycin I, II and III on the inhibition rate of transplanted
tumor of human non-small cell lung cancer cell A549 in nude mice (x̅ ± s)

| | | Number of | Body | Body | | Inhibition |
|---|---|---|---|---|---|---|
| | Dose | Animals | Weight (g) | Weight (g) | Tumor Weight | Rate |
| Group | (mg/kg) | (Start/End) | (d1) | (d30) | (g) | (%) |
| Model | 0 | 6/6 | 21.21 ± 1.34 | 24.57 ± 1.71 | 2.39 ± 0.24 | — |
| Group | | | | | | |
| Cyclophos- | 30 | 6/6 | 21.89 ± 1.22 | 19.27 ± 1.49 | 0.70 ± 0.39*** | 70.59 |
| phamide | | | | | | |
| Isovaleryl | 25 | 6/6 | 21.54 ± 1.27 | 24.20 ± 1.33 | 1.82 ± 0.19** | 24.00 |
| spiramycin | 50 | 6/6 | 21.48 ± 1.46 | 25.56 ± 1.67 | 1.00 ± 0.44*** | 58.10 |
| I | 100 | 6/6 | 21.36 ± 1.82 | 24.47 ± 1.98 | 0.73 ± 0.45*** | 69.52 |
| Isovaleryl | 25 | 6/6 | 20.91 ± 1.07 | 21.32 ± 1.88 | 1.90 ± 0.31* | 20.73 |
| spiramycin | 50 | 6/6 | 20.03 ± 1.88 | 12.92 ± 1.68 | 1.63 ± 0.32** | 31.87 |
| II | 100 | 6/6 | 21.71 ± 1.94 | 12.42 ± 1.37 | 0.95 ± 0.42*** | 60.19 |
| Isovaleryl | 25 | 6/6 | 21.68 ± 1.25 | 23.97 ± 1.01 | 1.49 ± 0.42* | 37.99 |
| spiramycin | 50 | 6/6 | 21.99 ± 1.40 | 24.36 ± 1.73 | 1.06 ± 0.49*** | 55.95 |
| III | 100 | 6/6 | 20.57 ± 2.28 | 23.53 ± 1.63 | 0.80 ± 0.43*** | 66.53 |

*p < 0.05 compared with the model group,
**p < 0.01 compared with the model group,
***p < 0.001 compared with the model group

TABLE 23
Effect of isovaleryl spiramycin I, II and III on the volume change of transplanted
tumor of human non-small cell lung cancer cell A549 in nude mice (x̅ ± s)

| | | | | | | Relative |
|---|---|---|---|---|---|---|
| | | | | | Relative | Tumor |
| | | Number of | Tumor | | Tumor | Proliferation |
| | Dose | Animals | Volume | Tumor Volume | Volume | Rate |
| Group | (mg/kg) | (Start/End) | (mm3) (d1) | (mm3) (d30) | (RTV) | (T/C) |
| Model | 0 | 6/6 | 118.12 ± 9.47 | 1901.00 ± 131.19 | 16.23 ± 2.25 | — |
| Group | | | | | | |
| Cyclophos- | 30 | 6/6 | 115.15 ± 10.00 | 733.89 ± 352.79*** | 9.47 ± 2.91** | 39.73 |
| phamide | | | | | | |
| Isovaleryl | 25 | 6/6 | 111.89 ± 12.17 | 1227.24 ± 329.84** | 10.91 ± 2.63** | 67.18 |
| spiramycin | 50 | 6/6 | 119.73 ± 5.73 | 750.44 ± 220.77*** | 6.40 ± 1.75*** | 39.42 |
| I | 100 | 6/6 | 110.27 ± 11.43 | 475.83 ± 355.37*** | 4.60 ± 3.69*** | 28.35 |
| Isovaleryl | 25 | 6/6 | 119.67 ± 7.25 | 1614.91 ± 65.26** | 13.54 ± 1.07* | 83.41 |
| spiramycin | 50 | 6/6 | 117.96 ± 4.88 | 1129.10 ± 326.54** | 9.54 ± 2.62*** | 58.75 |
| II | 100 | 6/6 | 116.96 ± 6.56 | 917.10 ± 270.86*** | 7.78 ± 2.03*** | 47.94 |
| Isovaleryl | 25 | 6/6 | 113.96 ± 6.85 | 1216.86 ± 295.20** | 10.70 ± 2.64** | 65.93 |
| spiramycin | 50 | 6/6 | 113.65 ± 11.57 | 856.65 ± 281.96*** | 7.67 ± 2.86*** | 47.25 |
| III | 100 | 6/6 | 118.69 ± 8.13 | 632.73 ± 350.89*** | 5.36 ± 3.01*** | 33.04 |

*p < 0.05 compared with the model group;
**p < 0.01 compared with the model group,
***p < 0.001 compared with the model group

11. Inhibition of Isovaleryl Spiramycin I, II and III on Human Glioma in Nude Mice Model

Establishment of a Mouse Solid Tumor Model

U251 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a cyclophosphamide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 24, and Table 25).

The tumor growth inhibition rates of isovaleryl spiramycin I groups with low, medium and high doses are 30.94%, 44.53% and 69.21%, respectively.

The tumor growth inhibition rates of isovaleryl spiramycin II groups with low, medium and high doses are 10.91%, 15.81% and 40.26%, respectively.

The tumor growth inhibition rates of isovaleryl spiramycin III groups with low, medium and high doses are 15.45%, 32.74% and 59.56%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 76.40%, 44.54% and 25.80%, respectively.

The relative tumor proliferation rates of isovaleryl spiramycin II groups with low, medium and high doses are 76.14%, 51.88% and 43.26%, respectively.

The relative tumor proliferation rates of isovaleryl spiramycin III groups with low, medium and high doses are 68.16%, 54.34% and 41.10%, respectively.

There are no significant changes in the mouse weight of isovaleryl spiramycin I, II and III groups with low, medium and high doses compared with the model group.

TABLE 24
Effect of isovaleryl spiramycin I, II and III on the inhibition rate of transplanted
tumor of human glioma cell U251 cells in nude mice (x̅ ± s)

| | | Number of | Body | Body | | Inhibition |
|---|---|---|---|---|---|---|
| | Dose | Animals | Weight (g) | Weight (g) | Tumor Weight | Rate |
| Group | (mg/kg) | (Start/End) | (d1) | (d30) | (g) | (%) |
| Model | 0 | 6/6 | 21.02 ± 0.37 | 20.50 ± 0.25 | 1.38 ± 0.05 | — |
| Group | | | | | | |
| Cyclophos- | 30 | 6/6 | 20.72 ± 0.48 | 19.40 ± 1.40 | 0.42 ± 0.06*** | 69.31 |
| phamide | | | | | | |
| Isovaleryl | 25 | 6/6 | 21.03 ± 0.55 | 21.09 ± 0.53 | 0.96 ± 0.08* | 30.94 |
| spiramycin | 50 | 6/6 | 20.78 ± 0.66 | 21.08 ± 0.54 | 0.77 ± 0.09** | 44.53 |
| I | 100 | 6/6 | 21.10 ± 0.35 | 20.86 ± 0.66 | 0.43 ± 0.01** | 69.21 |
| Isovaleryl | 25 | 6/6 | 20.77 ± 0.65 | 20.85 ± 0.79 | 1.23 ± 0.12 | 10.91 |
| spiramycin | 50 | 6/6 | 21.25 ± 0.46 | 20.86 ± 0.43 | 1.16 ± 0.10* | 15.81 |
| II | 100 | 6/6 | 20.92 ± 0.82 | 21.47 ± 0.70 | 0.83 ± 0.12*** | 40.26 |
| Isovaleryl | 25 | 6/6 | 20.96 ± 0.58 | 20.70 ± 0.51 | 1.17 ± 0.11** | 15.45 |
| spiramycin | 50 | 6/6 | 21.25 ± 0.53 | 21.10 ± 0.60 | 0.93 ± 0.12** | 32.74 |
| III | 100 | 6/6 | 21.33 ± 0.62 | 21.03 ± 0.55 | 0.56 ± 0.05*** | 59.56 |

*p < 0.05 compared with the model group,
**p < 0.01 compared with the model group,
***p < 0.001 compared with the model group

TABLE 25
Effect of isovaleryl spiramycin I, II and III on the volume change of transplanted
tumor of human glioma cell U251 cells in nude mice (x̅ ± s)

| | | | | | | Relative |
|---|---|---|---|---|---|---|
| | | | | | Relative | Tumor |
| | | Number of | Tumor | | Tumor | Proliferation |
| | Dose | Animals | Volume | Tumor Volume | Volume | Rate |
| Group | (mg/kg) | (Start/End) | (mm3) (d1) | (mm3) (d30) | (RTV) | (T/C) |
| Model | 0 | 6/6 | 125.74 ± 10.43 | 2082.09 ± 83.17 | 16.69 ± 2.03 | — |
| Group | | | | | | |
| Cyclophos- | 30 | 6/6 | 119.00 ± 11.13 | 504.56 ± 93.91** | 4.23 ± 0.95*** | 62.92 |
| phamide | | | | | | |
| Isovaleryl | 25 | 6/6 | 122.81 ± 10.35 | 1550.33 ± 72.99** | 12.69 ± 1.08** | 76.40 |
| spiramycin | 50 | 6/6 | 124.39 ± 10.31 | 930.42 ± 204.72** | 10.67 ± 1.09*** | 44.54 |
| I | 100 | 6/6 | 124.88 ± 6.57 | 537.02 ± 93.66*** | 10.05 ± 0.71*** | 25.80 |
| Isovaleryl | 25 | 6/6 | 125.90 ± 11.45 | 1596.45 ± 267.65* | 14.73 ± 1.33* | 76.14 |
| spiramycin | 50 | 6/6 | 118.33 ± 11.65 | 1030.10 ± 207.19** | 14.31 ± 0.98* | 51.88 |
| II | 100 | 6/6 | 122.74 ± 10.75 | 890.78 ± 225.34** | 12.88 ± 1.54** | 43.26 |
| Isovaleryl | 25 | 6/6 | 124.24 ± 10.18 | 1413.41 ± 221.90* | 13.47 ± 1.67** | 68.16 |
| spiramycin | 50 | 6/6 | 121.24 ± 9.15 | 1096.57 ± 173.49** | 12.63 ± 1.27*** | 54.34 |
| III | 100 | 6/6 | 120.86 ± 6.34 | 832.79 ± 182.98** | 10.96 ± 0.82*** | 41.10 |

*p < 0.05 compared with the model group;
**p < 0.01 compared with the model group,
***p < 0.001 compared with the model group

12. Inhibition of Isovaleryl Spiramycin I, II and III on Human Glioblastoma in Nude Mice Model

Establishment of a Mouse Solid Tumor Model

A172 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a temozolomide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 26, and Table 27).

The tumor growth inhibition rates of isovaleryl spiramycin I groups with low, medium and high doses are 30.75%, 44.26% and 68.79%, respectively.

The tumor growth inhibition rates of isovaleryl spiramycin II groups with low, medium and high doses are 10.85%, 15.71% and 40.01%, respectively.

The tumor growth inhibition rates of isovaleryl spiramycin III groups with low, medium and high doses are 15.35%, 32.54% and 59.19%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 74.49%, 43.43% and 25.16%, respectively.

The relative tumor proliferation rates of isovaleryl spiramycin II groups with low, medium and high doses are 74.24%, 50.59% and 42.18%, respectively.

The relative tumor proliferation rates of isovaleryl spiramycin III groups with low, medium and high doses are 66.45%, 52.89% and 40.08%, respectively.

There are no significant changes in the mouse weight of isovaleryl spiramycin I, II and III groups with low, medium and high doses compared with the model group.

TABLE 26
Effect of isovaleryl spiramycin I, II and III on the inhibition rate of transplanted
tumor of human glioblastoma cell A172 in nude mice (x̅ ± s)

| | | Number of | Body | Body | | Inhibition |
|---|---|---|---|---|---|---|
| | Dose | Animals | Weight (g) | Weight (g) | Tumor Weight | Rate |
| Group | (mg/kg) | (Start/End) | (d1) | (d30) | (g) | (%) |
| Model | 0 | 6/6 | 20.80 ± 0.66 | 20.50 ± 0.75 | 1.39 ± 0.05 | — |
| Group | | | | | | |
| Temozo- | 50 | 6/6 | 21.07 ± 0.57 | 19.83 ± 1.24 | 0.43 ± 0.06*** | 68.89 |
| lomide | | | | | | |
| Isovaleryl | 12.5 | 6/6 | 21.06 ± 0.43 | 20.34 ± 0.17 | 0.96 ± 0.08** | 30.75 |
| spiramycin | 25 | 6/6 | 20.98 ± 0.73 | 20.93 ± 0.54 | 0.77 ± 0.09** | 44.26 |
| I | 50 | 6/6 | 21.00 ± 0.65 | 20.88 ± 0.58 | 0.43 ± 0.07*** | 68.79 |
| Isovaleryl | 12.5 | 6/6 | 21.36 ± 0.67 | 21.36 ± 0.87 | 1.24 ± 0.12 | 10.85 |
| spiramycin | 25 | 6/6 | 21.10 ± 0.62 | 21.30 ± 0.89 | 1.17 ± 0.10* | 15.71 |
| II | 50 | 6/6 | 21.08 ± 0.74 | 20.97 ± 0.68 | 0.84 ± 0.12** | 40.01 |
| Isovaleryl | 12.5 | 6/6 | 21.54 ± 0.36 | 20.64 ± 0.54 | 1.18 ± 0.11* | 15.35 |
| spiramycin | 25 | 6/6 | 20.98 ± 0.47 | 21.54 ± 0.72 | 0.94 ± 0.12** | 32.54 |
| III | 50 | 6/6 | 21.59 ± 0.32 | 20.67 ± 0.56 | 0.57 ± 0.05*** | 59.19 |

*p < 0.05 compared with the model group,
**p < 0.01 compared with the model group,
***p < 0.001 compared with the model group

TABLE 27
Effect of isovaleryl spiramycin I, II and III on the volume change of transplanted
tumor of human glioblastoma cell A172 in nude mice (x̅ ± s)

| | | | | | | Relative |
|---|---|---|---|---|---|---|
| | | | | | Relative | Tumor |
| | | Number of | Tumor | | Tumor | Proliferation |
| | Dose | Animals | Volume | Tumor Volume | Volume | Rate |
| Group | (mg/kg) | (Start/End) | (mm3) (d1) | (mm3) (d30) | (RTV) | (T/C) |
| Model | 0 | 6/6 | 123.2 ± 10.22 | 2040.44 ± 81.50 | 16.69 ± 2.03 | — |
| Group | | | | | | |
| Temozo- | 50 | 6/6 | 116.6 ± 10.91 | 482.11 ± 89.74*** | 4.12 ± 2.03*** | 61.35 |
| lomide | | | | | | |
| Isovalery | 12.5 | 6/6 | 120.36 ± 10.15 | 1519.32 ± 71.53* | 12.69 ± 1.08** | 74.49 |
| 1 | 25 | 6/6 | 121.90 ± 10.10 | 889.01 ± 195.61** | 10.67 ± 1.08*** | 43.43 |
| spiramycin | 50 | 6/6 | 122.38 ± 6.44 | 513.12 ± 89.49*** | 10.05 ± 0.71*** | 25.16 |
| I | | | | | | |
| Isovalery | 12.5 | 6/6 | 123.39 ± 11.22 | 1525.41 ± 255.74* | 14.73 ± 1.33* | 74.24 |
| 1 | 25 | 6/6 | 115.96 ± 11.42 | 984.26 ± 197.97** | 14.31 ± 0.98* | 50.59 |
| spiramycin | 50 | 6/6 | 120.28 ± 10.53 | 851.14 ± 215.31** | 12.88 ± 1.54** | 42.18 |
| II | | | | | | |
| Isovalery | 12.5 | 6/6 | 121.75 ± 9.98 | 1305.51 ± 212.02* | 13.47 ± 1.67** | 66.45 |
| 1 | 25 | 6/6 | 118.81 ± 8.97 | 1047.77 ± 165.77** | 12.63 ± 1.27*** | 52.89 |
| spiramycin | 50 | 6/6 | 118.44 ± 6.21 | 795.73 ± 174.84*** | 10.96 ± 0.82*** | 40.08 |
| III | | | | | | |

*p < 0.05 compared with the model group;
**p < 0.01 compared with the model group,
***p < 0.001 compared with the model group

13. Inhibition of Isovaleryl Spiramycin I, II and III on Human Lymphoma in Nude Mice Model

Establishment of a Mouse Solid Tumor Model

U937 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a cyclophosphamide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 28, and Table 29).

The tumor growth inhibition rates of isovaleryl spiramycin I groups with low, medium and high doses are 41.73%, 50.73% and 65.03%, respectively.

The tumor growth inhibition rates of isovaleryl spiramycin II groups with low, medium and high doses are 25.61%, 35.44% and 43.63%, respectively.

The tumor growth inhibition rates of isovaleryl spiramycin III groups with low, medium and high doses are 41.19%, 53.03% and 61.77%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 68.65%, 39.74% and 35.25%, respectively.

The relative tumor proliferation rates of isovaleryl spiramycin II groups with low, medium and high doses are 74.19%, 52.10% and 46.09%, respectively.

The relative tumor proliferation rates of isovaleryl spiramycin III groups with low, medium and high doses are 63.36%, 49.30% and 38.66%, respectively.

There are no significant changes in the mouse weight of isovaleryl spiramycin I, II and III groups with low, medium and high doses compared with the model group.

TABLE 28
Effect of isovaleryl spiramycin I, II and III on the inhibition rate of transplanted
tumor of human lymphoma cell U937 in nude mice (x̅ ± s)

| | | Number of | Body | Body | | Inhibition |
|---|---|---|---|---|---|---|
| | Dose | Animals | Weight (g) | Weight (g) | Tumor Weight | Rate |
| Group | (mg/kg) | (Start/End) | (d1) | (d30) | (g) | (%) |
| Model | 0 | 6/6 | 20.77 ± 0.51 | 21.11 ± 0.53 | 1.94 ± 0.27 | — |
| Group | | | | | | |
| Cyclophos- | 30 | 6/6 | 21.00 ± 0.72 | 19.72 ± 0.95 | 0.68 ± 0.12*** | 65.11 |
| phamide | | | | | | |
| Isovaleryl | 12.5 | 6/6 | 21.19 ± 0.66 | 21.16 ± 0.74 | 1.13 ± 0.60* | 41.73 |
| spiramycin | 25 | 6/6 | 21.28 ± 0.52 | 21.01 ± 0.68 | 0.95 ± 0.42** | 50.73 |
| I | 50 | 6/6 | 20.64 ± 0.39 | 20.94 ± 0.77 | 0.68 ± 0.09*** | 65.03 |
| Isovaleryl | 12.5 | 6/6 | 21.10 ± 0.64 | 20.97 ± 0.74 | 1.44 ± 0.64 | 25.61 |
| spiramycin | 25 | 6/6 | 21.05 ± 0.30 | 21.03 ± 0.632 | 1.25 ± 0.66* | 35.44 |
| II | 50 | 6/6 | 20.80 ± 0.51 | 21.22 ± 0.51 | 1.09 ± 0.62* | 43.63 |
| Isovaleryl | 12.5 | 6/6 | 20.84 ± 0.68 | 20.92 ± 0.63 | 1.14 ± 0.54* | 41.19 |
| spiramycin | 25 | 6/6 | 20.91 ± 0.58 | 20.74 ± 0.352 | 0.91 ± 0.51** | 53.03 |
| III | 50 | 6/6 | 21.16 ± 0.61 | 21.24 ± 0.50 | 0.74 ± 0.18*** | 61.77 |

*p < 0.05 compared with the model group,
**p < 0.01 compared with the model group,
***p < 0.001 compared with the model group

TABLE 29
Effect of isovaleryl spiramycin I, II and III on the volume change of transplanted
tumor of human lymphoma cell U937 in nude mice (x̅ ± s)

| | | | | | | Relative |
|---|---|---|---|---|---|---|
| | | | | | Relative | Tumor |
| | | Number of | Tumor | | Tumor | Proliferation |
| | Dose | Animals | Volume | Tumor Volume | Volume | Rate |
| Group | (mg/kg) | (Start/End) | (mm3) (d1) | (mm3) (d30) | (RTV) | (T/C) |
| Model | 0 | 6/6 | 115.70 ± 9.96 | 1986.59 ± 516.10 | 17.04 ± 4.02 | — |
| Group | | | | | | |
| Cyclophos- | 30 | 6/6 | 125.00 ± 32.61 | 722.72 ± 55.32** | 6.04 ± 1.26*** | 35.44 |
| phamide | | | | | | |
| Isovaleryl | 12.5 | 6/6 | 108.43 ± 6.49 | 1270.43 ± 435.28* | 11.70 ± 3.59* | 68.65 |
| spiramycin | 25 | 6/6 | 108.61 ± 9.06 | 734.22 ± 76.87** | 6.77 ± 0.57** | 39.74 |
| I | 50 | 6/6 | 109.98 ± 5.88 | 660.17 ± 37.49** | 6.01 ± 2.26** | 35.25 |
| Isovaleryl | 12.5 | 6/6 | 111.58 ± 23.26 | 1400.95 ± 558.82 | 12.64 ± 5.00 | 74.19 |
| spiramycin | 25 | 6/6 | 120.21 ± 24.68 | 1117.59 ± 565.29* | 8.88 ± 2.64** | 52.10 |
| II | 50 | 6/6 | 111.50 ± 7.03 | 885.75 ± 418.53** | 7.85 ± 3.27** | 46.09 |
| Isovaleryl | 12.5 | 6/6 | 119.70 ± 18.30 | 1314.25 ± 479.76* | 10.80 ± 3.02* | 63.36 |
| spiramycin | 25 | 6/6 | 112.93 ± 27.42 | 939.79 ± 296.72** | 8.40 ± 2.25** | 49.30 |
| III | 50 | 6/6 | 112.85 ± 21.90 | 728.49 ± 51.66** | 6.59 ± 0.90** | 38.66 |

*p < 0.05 compared with the model group;
**p < 0.01 compared with the model group,
***p < 0.001 compared with the model group

14. Inhibition of Isovaleryl Spiramycin I, II and III on Human Cervical Cancer in Nude Mice Model

Establishment of a Mouse Solid Tumor Model

HeLa cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a cyclophosphamide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 30, and Table 31).

The tumor growth inhibition rates of isovaleryl spiramycin I groups with low, medium and high doses are 46.69%, 51.57% and 65.55%, respectively.

The tumor growth inhibition rates of isovaleryl spiramycin II groups with low, medium and high doses are 30.67%, 42.90% and 43.30%, respectively.

The tumor growth inhibition rates of isovaleryl spiramycin III groups with low, medium and high doses are 39.33%, 52.41% and 61.68%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 69.18%, 39.57% and 30.91%, respectively.

The relative tumor proliferation rates of isovaleryl spiramycin II groups with low, medium and high doses are 30.67%, 42.90% and 43.30%, respectively.

The relative tumor proliferation rates of isovaleryl spiramycin III groups with low, medium and high doses are 39.33%, 52.41% and 61.68%, respectively.

There are no significant changes in the mouse weight of isovaleryl spiramycin I, II and III groups with low, medium and high doses compared with the model group.

TABLE 30
Effect of isovaleryl spiramycin I, II and III on the inhibition rate of transplanted
tumor of human cervical cancer cell HeLa in nude mice (x̅ ± s)

| | | Number of | Body | Body | | Inhibition |
|---|---|---|---|---|---|---|
| | Dose | Animals | Weight (g) | Weight (g) | Tumor Weight | Rate |
| Group | (mg/kg) | (Start/End) | (d1) | (d30) | (g) | (%) |
| Model | 0 | 6/6 | 21.33 ± 0.48 | 21.27 ± 0.68 | 1.93 ± 0.21 | — |
| Group | | | | | | |
| Cyclophos- | 30 | 6/6 | 21.10 ± 0.69 | 19.73 ± 1.22 | 0.66 ± 0.13*** | 65.74 |
| phamide | | | | | | |
| Isovaleryl | 12.5 | 6/6 | 20.72 ± 0.49 | 21.10 ± 0.68 | 1.03 ± 0.59* | 46.69 |
| spiramycin | 25 | 6/6 | 21.07 ± 0.44 | 21.23 ± 0.73 | 0.94 ± 0.48** | 51.57 |
| I | 50 | 6/6 | 20.82 ± 0.72 | 20.95 ± 0.45 | 0.67 ± 0.09*** | 65.55 |
| Isovaleryl | 12.5 | 6/6 | 20.92 ± 0.46 | 21.16 ± 0.51 | 1.34 ± 0.60 | 30.67 |
| spiramycin | 25 | 6/6 | 20.87 ± 0.34 | 21.25 ± 0.55 | 1.10 ± 0.62* | 42.90 |
| II | 50 | 6/6 | 20.77 ± 0.43 | 21.09 ± 0.36 | 1.10 ± 0.61* | 43.30 |
| Isovaleryl | 12.5 | 6/6 | 21.30 ± 0.64 | 20.67 ± 0.45 | 1.17 ± 0.64* | 39.33 |
| spiramycin | 25 | 6/6 | 20.87 ± 0.78 | 20.80 ± 0.55 | 0.92 ± 0.49** | 52.41 |
| III | 50 | 6/6 | 21.09 ± 0.51 | 21.10 ± 0.52 | 0.75 ± 0.11*** | 61.68 |

*p < 0.05 compared with the model group,
**p < 0.01 compared with the model group,
***p < 0.001 compared with the model group

TABLE 31
Effect of isovaleryl spiramycin I, II and III on the volume change of transplanted
tumor of human cervical cancer cell HeLa in nude mice (x̅ ± s)

| | | | | | | Relative |
|---|---|---|---|---|---|---|
| | | | | | Relative | Tumor |
| | | Number of | Tumor | | Tumor | Proliferation |
| | Dose | Animals | Volume | Tumor Volume | Volume | Rate |
| Group | (mg/kg) | (Start/End) | (mm3) (d1) | (mm3) (d30) | (RTV) | (T/C) |
| Model | 0 | 6/6 | 119.20 ± 11.23 | 1991.26 ± 516.28 | 16.58 ± 3.86 | — |
| Group | | | | | | |
| Cyclophos- | 30 | 6/6 | 127.17 ± 35.82 | 724.89 ± 53.90** | 6.00 ± 1.35*** | 36.17 |
| phamide | | | | | | |
| Isovaleryl | 12.5 | 6/6 | 112.43 ± 6.87 | 1287.77 ± 552.57* | 11.47 ± 4.97 | 69.18 |
| spiramycin | 25 | 6/6 | 112.78 ± 9.72 | 738.39 ± 75.32** | 6.56 ± 0.55** | 39.57 |
| I | 50 | 6/6 | 114.15 ± 6.08 | 588.18 ± 73.34** | 5.13 ± 0.68*** | 30.91 |
| Isovaleryl | 12.5 | 6/6 | 115.41 ± 23.27 | 1301.28 ± 391.77* | 11.36 ± 2.90 | 68.55 |
| spiramycin | 25 | 6/6 | 125.04 ± 20.71 | 1184.42 ± 369.49* | 9.30 ± 1.74** | 56.07 |
| II | 50 | 6/6 | 111.84 ± 4.83 | 851.58 ± 93.66** | 7.62 ± 0.85** | 45.96 |
| Isovaleryl | 12.5 | 6/6 | 123.36 ± 21.45 | 1281.92 ± 242.71* | 1.43 ± 1.47** | 62.92 |
| spiramycin | 25 | 6/6 | 115.2 ± 27.18 | 1007.46 ± 481.36** | 8.63 ± 3.37** | 52.05 |
| III | 50 | 6/6 | 113.35 ± 20.84 | 727.16 ± 46.92** | 6.52 ± 0.73** | 39.33 |

*p < 0.05 compared with the model group;
**p < 0.01 compared with the model group,
***p < 0.001 compared with the model group

15. Inhibition of Isovaleryl Spiramycin I, II and III on Human Renal Clear Cell Adenocarcinoma in Nude Mice Model

Establishment of a Mouse Solid Tumor Model

786-O cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a cyclophosphamide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 32, and Table 33).

The tumor growth inhibition rates of isovaleryl spiramycin I groups with low, medium and high doses are 34.70%, 39.22% and 64.36%, respectively.

The tumor growth inhibition rates of isovaleryl spiramycin II groups with low, medium and high doses are 19.69%, 41.09% and 60.00%, respectively.

The tumor growth inhibition rates of isovaleryl spiramycin III groups with low, medium and high doses are 35.80%, 52.14% and 62.49%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 37.88%, 37.19% and 36.89%, respectively.

The relative tumor proliferation rates of isovaleryl spiramycin II groups with low, medium and high doses are 76.92%, 53.61% and 35.74%, respectively.

The relative tumor proliferation rates of isovaleryl spiramycin III groups with low, medium and high doses are 73.13%, 51.33% and 34.20%, respectively.

There are no significant changes in the mouse weight of isovaleryl spiramycin I, II and III groups with low, medium and high doses compared with the model group.

TABLE 32
Effect of isovaleryl spiramycin I, II and III on the inhibition rate of transplanted
tumor of human renal clear cell adenocarcinoma cell 786-O in nude mice (x̅ ± s)

| | | Number of | Body | Body | | Inhibition |
|---|---|---|---|---|---|---|
| | Dose | Animals | Weight (g) | Weight (g) | Tumor Weight | Rate |
| Group | (mg/kg) | (Start/End) | (d1) | (d30) | (g) | (%) |
| Model | 0 | 6/6 | 21.83 ± 1.32 | 24.17 ± 1.29 | 2.14 ± 0.07 | — |
| Group | | | | | | |
| Cyclophos- | 30 | 6/6 | 21.58 ± 0.81 | 19.42 ± 0.67 | 0.63 ± 0.04*** | 70.51 |
| phamide | | | | | | |
| Isovaleryl | 25 | 6/6 | 21.87 ± 0.89 | 22.95 ± 0.88 | 1.40 ± 0.40** | 34.70 |
| spiramycin | 50 | 6/6 | 22.00 ± 0.94 | 22.82 ± 0.64 | 1.31 ± 0.35*** | 39.22 |
| I | 100 | 6/6 | 21.88 ± 0.54 | 23.00 ± 0.67 | 0.76 ± 0.13*** | 64.36 |
| Isovaleryl | 25 | 6/6 | 22.05 ± 0.71 | 23.33 ± 0.54 | 1.72 ± 0.27** | 19.69 |
| spiramycin | 50 | 6/6 | 22.42 ± 0.54 | 23.42 ± 0.50 | 1.26 ± 0.31** | 41.09 |
| II | 100 | 6/6 | 22.33 ± 0.41 | 23.45 ± 0.91 | 0.85 ± 0.16*** | 60.00 |
| Isovaleryl | 25 | 6/6 | 22.72 ± 0.78 | 23.85 ± 0.89 | 1.38 ± 0.42** | 35.80 |
| spiramycin | 50 | 6/6 | 21.43 ± 1.08 | 22.33 ± 1.09 | 1.02 ± 0.27*** | 52.14 |
| III | 100 | 6/6 | 22.28 ± 1.72 | 23.43 ± 1.69 | 0.80 ± 0.05*** | 62.49 |

*p < 0.05 compared with the model group,
**p < 0.01 compared with the model group,
***p < 0.001 compared with the model group

TABLE 33
Effect of isovaleryl spiramycin I, II and III on the volume change of transplanted tumor
of human renal clear cell adenocarcinoma cell 786-O in nude mice (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm 3) (d 30) | Volume (RTV) | Rate (T/C) |
| Model Group | 0 | 6/6 | 114.09 ± 9.07 | 2060.98 ± 168.33 | 18.06 ± 1.78 | — |
| Cyclophosphamide | 30 | 6/6 | 108.64 ± 14.40 | 734.23 ± 24.66*** | 6.76 ± 0.79*** | 37.41 |
| Isovaleryl | 25 | 6/6 | 96.85 ± 9.98 | 677.21 ± 58.76*** | 6.99 ± 0.83*** | 37.88 |
| spiramycin I | 50 | 6/6 | 96.82 ± 8.52 | 664.44 ± 73.49*** | 6.86 ± 1.01*** | 37.19 |
| | 100 | 6/6 | 103.25 ± 9.71 | 702.74 ± 37.66*** | 6.81 ± 0.31*** | 36.87 |
| Isovaleryl | 25 | 6/6 | 96.82 ± 8.52 | 1415.80 ± 201.11* | 14.62 ± 1.93* | 76.92 |
| spiramycin II | 50 | 6/6 | 96.82 ± 8.52 | 982.68 ± 265.02* | 10.19 ± 2.62** | 53.61 |
| | 100 | 6/6 | 103.26 ± 9.71 | 701.57 ± 127.91** | 6.79 ± 1.65*** | 35.74 |
| Isovaleryl | 25 | 6/6 | 106.47 ± 6.86 | 1479.90 ± 458.97* | 18.90 ± 3.60 | 73.13 |
| spiramycin III | 50 | 6/6 | 96.82 ± 8.52 | 944.60 ± 112.60** | 9.76 ± 1.44** | 51.33 |
| | 100 | 6/6 | 103.26 ± 9.71 | 635.26 ± 78.22*** | 6.18 ± 0.61*** | 34.20 |

*p < 0.05 compared with the model group;
**p < 0.01 compared with the model group,
***p < 0.001 compared with the model group

16. Inhibition of Isovaleryl Spiramycin I, II and III on Human Renal Cell Adenocarcinoma in Nude Mice Model

Establishment of a Mouse Solid Tumor Model

769-P cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: a model group, a cyclophosphamide group, isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 34, and Table 35).

The tumor growth inhibition rates of isovaleryl spiramycin I groups with low, medium and high doses are 33.68%, 47.33% and 68.82%, respectively.

The tumor growth inhibition rates of isovaleryl spiramycin II groups with low, medium and high doses are 38.29%, 47.61% and 61.79%, respectively.

The tumor growth inhibition rates of isovaleryl spiramycin III groups with low, medium and high doses are 40.87%, 60.50% and 64.46%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 75.78%, 57.12% and 36.88%, respectively.

The relative tumor proliferation rates of isovaleryl spiramycin II groups with low, medium and high doses are 68.22%, 42.64% and 34.76%, respectively.

The relative tumor proliferation rates of isovaleryl spiramycin III groups with low, medium and high doses are 61.59%, 51.59% and 35.55%, respectively.

There are no significant changes in the mouse weight of isovaleryl spiramycin I, II and III groups with low, medium and high doses compared with the model group.

TABLE 34
Effect of isovaleryl spiramycin I, II and III on the inhibition rate of transplanted
tumor of human renal cell adenocarcinoma cell 769-P in nude mice (x̅ ± s)

| | | Number of | Body | Body | | |
|---|---|---|---|---|---|---|
| | Dose | Animals | Weight (g) | Weight (g) | Tumor | Inhibition |
| Group | (mg/kg) | (Start/End) | (d 1) | (d 30) | Weight (g) | Rate (%) |
| Model Group | 0 | 6/6 | 21.79 ± 1.17 | 21.83 ± 1.32 | 2.06 ± 0.08*** | — |
| Cyclophosphamide | 30 | 6/6 | 21.61 ± 1.00 | 21.58 ± 0.81 | 0.6 ± 0.03*** | 70.52 |
| Isovaleryl | 25 | 6/6 | 21.44 ± 1.45 | 21.87 ± 0.89 | 1.37 ± 0.55* | 33.68 |
| spiramycin I | 50 | 6/6 | 21.79 ± 1.37 | 22.00 ± 0.94 | 1.09 ± 0.48** | 47.33 |
| | 100 | 6/6 | 21.39 ± 1.24 | 21.88 ± 0.54 | 0.64 ± 0.01*** | 68.82 |
| Isovaleryl | 25 | 6/6 | 22.17 ± 1.47 | 22.05 ± 0.71 | 1.27 ± 0.42** | 38.29 |
| spiramycin II | 50 | 6/6 | 22.07 ± 1.52 | 22.42 ± 0.54 | 1.3 ± 0.23*** | 47.61 |
| | 100 | 6/6 | 21.96 ± 1.19 | 22.33 ± 0.41 | 0.79 ± 0.06*** | 61.79 |
| Isovaleryl | 25 | 6/6 | 23.09 ± 1.90 | 22.72 ± 0.78 | 1.22 ± 0.46** | 40.87 |
| spiramycin III | 50 | 6/6 | 22.90 ± 1.00 | 21.43 ± 1.08 | 0.82 ± 0.06*** | 60.50 |
| | 100 | 6/6 | 20.49 ± 1.14 | 22.28 ± 1.72 | 0.73 ± 0.08*** | 64.46 |

*p < 0.05 compared with the model group,
**p < 0.01 compared with the model group,
***p < 0.001 compared with the model group

TABLE 35
Effect of isovaleryl spiramycin I, II and III on the volume change of transplanted
tumor of human renal cell adenocarcinoma cell 769-P in nude mice (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3) (d 30) | Volume (RTV) | Rate (T/C) |
| Model Group | 0 | 6/6 | 112.53 ± 10.69 | 2076.78 ± 168.33 | 18.45 ± 1.79 | — |
| Cyclophosphamide | 30 | 6/6 | 107.92 ± 15.94 | 704.00 ± 45.50*** | 6.52 ± 0.90*** | 35.34 |
| Isovaleryl | 25 | 6/6 | 106.47 ± 6.86 | 1489.03 ± 450.17* | 13.99 ± 3.52 | 75.78 |
| spiramycin I | 50 | 6/6 | 96.82 ± 8.51 | 1020.75 ± 282.92* | 10.54 ± 2.65* | 57.12 |
| | 100 | 6/6 | 103.26 ± 9.71 | 702.74 ± 37.66*** | 6.81 ± 0.30*** | 36.88 |
| Isovaleryl | 25 | 6/6 | 162.50 ± 59.24 | 2045.85 ± 301.35 | 12.59 ± 5.32 | 68.22 |
| spiramycin II | 50 | 6/6 | 193.10 ± 35.41 | 1519.68 ± 239.38** | 7.87 ± 2.29*** | 42.64 |
| | 100 | 6/6 | 109.14 ± 10.13 | 700.14 ± 100.08*** | 6.42 ± 0.99*** | 34.76 |
| Isovaleryl | 25 | 6/6 | 146.32 ± 43.02 | 1663.32 ± 445.50* | 11.37 ± 1.469* | 61.59 |
| spiramycin III | 50 | 6/6 | 180.43 ± 51.70 | 1717.76 ± 524.33** | 9.52 ± 1.67** | 51.59 |
| | 100 | 6/6 | 104.38 ± 12.92 | 684.88 ± 62.28*** | 6.56 ± 0.28*** | 36.55 |

*p < 0.05 compared with the model group;
**p < 0.01 compared with the model group,
***p < 0.001 compared with the model group

17. Inhibition of Isovaleryl Spiramycin I, II and III on Human HT-29 in Nude Mice Model

Establishment of a Mouse Solid Tumor Model

HT-29 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 36, and Table 37).

The tumor growth inhibition rates of isovaleryl spiramycin I groups with low, medium and high doses are 16.10%, 49.72% and 70.14%, respectively.

The tumor growth inhibition rates of isovaleryl spiramycin II groups with low, medium and high doses are 16.24%, 32.41% and 55.74%, respectively.

The tumor growth inhibition rates of isovaleryl spiramycin III groups with low, medium and high doses are 19.22%, 41.35% and 63.19%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 81.60%, 49.22% and 29.11%, respectively.

The relative tumor proliferation rates of isovaleryl spiramycin II groups with low, medium and high doses are 84.18%, 79.34% and 44.05%, respectively.

The relative tumor proliferation rates of isovaleryl spiramycin III groups with low, medium and high doses are 85.54%, 53.48% and 35.63%, respectively.

There are no significant changes in the mouse weight of isovaleryl spiramycin I, II and III groups with low, medium and high doses compared with the model group.

TABLE 36
Effect of isovaleryl spiramycin I, II and III on the inhibition rate of transplanted
tumor of HT-29 cells in nude mice (x̅ ± s)

| | | Number of | Body | Body | | |
|---|---|---|---|---|---|---|
| | Dose | Animals | Weight (g) | Weight (g) | Tumor | Inhibition |
| Group | (mg/kg) | (Start/End) | (d 1) | (d 30) | Weight (g) | Rate (%) |
| Model Group | 0 | 6/6 | 21.29 ± 1.40 | 21.04 ± 1.39 | 2.35 ± 0.44 | |
| Cyclophosphamide | 30 | 6/6 | 21.42 ± 1.35 | 18.69 ± 1.21 | 0.50 ± 0.25*** | 78.87 |
| Isovaleryl | 25 | 6/6 | 21.41 ± 1.41 | 20.98 ± 0.99 | 1.97 ± 0.31 | 16.10 |
| spiramycin I | 50 | 6/6 | 21.31 ± 1.53 | 20.56 ± 1.09 | 1.18 ± 0.34*** | 49.72 |
| | 100 | 6/6 | 20.77 ± 1.13 | 21.19 ± 1.26 | 0.70 ± 0.29*** | 70.14 |
| Isovaleryl | 25 | 6/6 | 21.04 ± 1.28 | 20.88 ± 1.29 | 1.97 ± 0.34 | 16.24 |
| spiramycin II | 50 | 6/6 | 21.36 ± 1.35 | 20.9 ± 1.29 | 1.59 ± 0.49* | 32.41 |
| | 100 | 6/6 | 20.80 ± 0.89 | 21.53 ± 1.08 | 1.04 ± 0.40*** | 55.74 |
| Isovaleryl | 25 | 6/6 | 21.31 ± 1.23 | 21.19 ± 1.52 | 1.90 ± 0.39 | 19.22 |
| spiramycin III | 50 | 6/6 | 21.22 ± 1.24 | 21.00 ± 0.98 | 1.38 ± 0.37** | 41.35 |
| | 100 | 6/6 | 21.34 ± 1.78 | 21.09 ± 1.43 | 0.87 ± 0.34*** | 63.19 |

*p < 0.05 compared with the model group,
**p < 0.01 compared with the model group,
***p < 0.001 compared with the model group

TABLE 37
Effect of isovaleryl spiramycin I, II and III on the volume change of transplanted
tumor of HT-29 cells in nude mice (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3) (d 30) | Volume (RTV) | Rate (T/C) |
| Model Group | 0 | 6/6 | 144.17 ± 35.58 | 1976.26 ± 294.65 | 14.41 ± 3.95 | |
| Cyclophosphamide | 30 | 6/6 | 176.59 ± 36.81 | 477.12 ± 166.46*** | 2.86 ± 1.44*** | 19.84 |
| Isovaleryl | 25 | 6/6 | 162.77 ± 38.95 | 1803.98 ± 271.55 | 11.76 ± 4.03 | 81.60 |
| spiramycin I | 50 | 6/6 | 157.41 ± 37.31 | 1109.16 ± 320.27** | 7.09 ± 1.47* | 49.22 |
| | 100 | 6/6 | 169.25 ± 35.39 | 648.93 ± 198.53*** | 4.19 ± 2.23** | 29.11 |
| Isovaleryl | 25 | 6/6 | 163.26 ± 38.80 | 1885.85 ± 219.49* | 12.13 ± 3.40 | 84.18 |
| spiramycin II | 50 | 6/6 | 142.56 ± 22.44 | 1568.80 ± 603.77* | 11.43 ± 5.13 | 79.34 |
| | 100 | 6/6 | 156.45 ± 37.20 | 921.49 ± 299.38*** | 6.35 ± 2.87** | 44.05 |
| Isovaleryl | 25 | 6/6 | 154.49 ± 33.07 | 1803.16 ± 368.50* | 12.32 ± 4.51 | 85.54 |
| spiramycin III | 50 | 6/6 | 161.76 ± 30.69 | 1252.64 ± 404.06* | 7.70 ± 1.65* | 53.48 |
| | 100 | 6/6 | 160.38 ± 38.86 | 826.76 ± 259.70*** | 5.13 ± 1.13** | 35.63 |

*p < 0.05 compared with the model group;
**p < 0.01 compared with the model group,
***p < 0.001 compared with the model group

18. Inhibition of Isovaleryl Spiramycin I, II and III on Human HL-60 in Nude Mice Model

Establishment of a Mouse Solid Tumor Model

HL-60 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 38, and Table 39).

The tumor growth inhibition rates of isovaleryl spiramycin I groups with low, medium and high doses are 11.57%, 34.78% and 64.19%, respectively.

The tumor growth inhibition rates of isovaleryl spiramycin II groups with low, medium and high doses are 15.92%, 29.48% and 51.81%, respectively.

The tumor growth inhibition rates of isovaleryl spiramycin III groups with low, medium and high doses are 17.10%, 29.92% and 55.05%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 84.96%, 59.54% and 32.70%, respectively.

The relative tumor proliferation rates of isovaleryl spiramycin II groups with low, medium and high doses are 75.39%, 65.18% and 41.36%, respectively.

The relative tumor proliferation rates of isovaleryl spiramycin III groups with low, medium and high doses are 76.09%, 61.90% and 39.87%, respectively.

There are no significant changes in isovaleryl spiramycin I, II and III groups with mouse weight of the low, medium and high doses compared with the model group.

TABLE 38
Effect of isovaleryl spiramycin I, II and III on the inhibition rate of transplanted
tumor of HL60 cells in nude mice (x̅ ± s)

| | | Number of | Body | Body | | |
|---|---|---|---|---|---|---|
| | Dose | Animals | Weight (g) | Weight (g) | Tumor | Inhibition |
| Group | (mg/kg) | (Start/End) | (d 1) | (d 30) | Weight (g) | Rate (%) |
| Model Group | 0 | 6/6 | 20.85 ± 1.36 | 20.71 ± 1.39 | 2.26 ± 0.34 | |
| Cyclophosphamide | 30 | 6/6 | 20.65 ± 1.47 | 18.47 ± 0.91 | 0.67 ± 0.30*** | 70.30 |
| Isovaleryl | 25 | 6/6 | 20.75 ± 1.20 | 20.66 ± 1.08 | 2.00 ± 0.34 | 11.57 |
| spiramycin I | 50 | 6/6 | 20.4 ± 1.60 | 20.35 ± 1.20 | 1.48 ± 0.38** | 34.78 |
| | 100 | 6/6 | 20.63 ± 1.33 | 20.56 ± 1.77 | 0.81 ± 0.42*** | 64.19 |
| Isovaleryl | 25 | 6/6 | 20.78 ± 1.31 | 20.59 ± 1.54 | 1.90 ± 0.33 | 15.92 |
| spiramycin II | 50 | 6/6 | 21.01 ± 1.20 | 20.72 ± 1.29 | 1.60 ± 0.45* | 29.48 |
| | 100 | 6/6 | 20.65 ± 1.27 | 20.32 ± 1.55 | 1.09 ± 0.34*** | 51.81 |
| Isovaleryl | 25 | 6/6 | 20.60 ± 1.57 | 20.67 ± 1.51 | 1.88 ± 0.25* | 17.10 |
| spiramycin III | 50 | 6/6 | 20.82 ± 1.42 | 20.59 ± 1.36 | 1.59 ± 0.45* | 29.92 |
| | 100 | 6/6 | 20.2l ± l .87 | 20.25 ± 1.22 | 1.01 ± 0.41*** | 55.05 |

*p < 0.05 compared with the model group,
**p < 0.01 compared with the model group,
***p < 0.001 compared with the model group

TABLE 39
Effect of isovaleryl spiramycin I, II and III on the volume change of transplanted
tumor of HL60 cells in nude mice (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3) (d 30) | Volume (RTV) | Rate (T/C) |
| Model Group | 0 | 6/6 | 186.92 ± 35.21 | 2009.89 ± 276.25 | 11.25 ± 3.65 | |
| Cyclophosphamide | 30 | 6/6 | 180.63 ± 29.97 | 466.98 ± 255.88*** | 2.78 ± 1.89** | 24.69 |
| Isovaleryl | 25 | 6/6 | 182.56 ± 25.80 | 1702.01 ± 305.75 | 9.56 ± 2.41 | 84.96 |
| spiramycin I | 50 | 6/6 | 189.04 ± 24.48 | 1230.70 ± 332.94** | 6.70 ± 2.58* | 59.54 |
| | 100 | 6/6 | 183.82 ± 20.41 | 657.95 ± 411.53*** | 3.68 ± 2.51** | 32.70 |
| Isovaleryl | 25 | 6/6 | 189.51 ± 28.58 | 1596.76 ± 293.76* | 8.48 ± 1.37 | 75.39 |
| spiramycin II | 50 | 6/6 | 184.06 ± 32.38 | 1344.04 ± 466.62* | 7.33 ± 2.36 | 65.18 |
| | 100 | 6/6 | 184.80 ± 33.56 | 834.34 ± 308.27*** | 4.65 ± 1.77** | 41.36 |
| Isovaleryl | 25 | 6/6 | 184.12 ± 22.53 | 1543.27 ± 190.73* | 8.56 ± 1.82 | 76.09 |
| spiramycin III | 50 | 6/6 | 189.31 ± 24.59 | 1308.87 ± 436.30* | 6.96 ± 2.25* | 61.90 |
| | 100 | 6/6 | 188.01 ± 27.66 | 789.65 ± 404.78*** | 4.48 ± 2.94** | 39.87 |

*p < 0.05 compared with the model group;
**p < 0.01 compared with the model group,
***p < 0.001 compared with the model group

19. Inhibition of Isovaleryl Spiramycin I, II and III on Human Thyroid Cancer in Nude Mice Model

Establishment of a Mouse Solid Tumor Model

TPC-1 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 40, and Table 41).

The tumor growth inhibition rates of isovaleryl spiramycin I groups with low, medium and high doses are 23.88%, 57.28% and 67.49%, respectively.

The tumor growth inhibition rates of isovaleryl spiramycin II groups with low, medium and high doses are 20.91%, 31.61% and 62.04%, respectively.

The tumor growth inhibition rates of isovaleryl spiramycin III groups with low, medium and high doses are 39.06%, 60.25% and 62.94%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 65.45%, 43.43% and 28.11%, respectively.

The relative tumor proliferation rates of isovaleryl spiramycin II groups with low, medium and high doses are 86.15%, 59.08% and 38.61%, respectively.

The relative tumor proliferation rates of isovaleryl spiramycin III groups with low, medium and high doses are 61.93%, 45.01% and 34.75%, respectively.

There are no significant changes in the mouse weight of isovaleryl spiramycin I, II and III groups with low, medium and high doses compared with the model group.

TABLE 40
Effect of isovaleryl spiramycin I, II and III on the inhibition rate of transplanted
tumor of human thyroid cancer cell TPC-1 in nude mice (x̅ ± s)

| | | Number of | Body | Body | | |
|---|---|---|---|---|---|---|
| | Dose | Animals | Weight (g) | Weight (g) | Tumor | Inhibition |
| Group | (mg/kg) | (Start/End) | (d 1) | (d 30) | Weight (g) | Rate (%) |
| Model Group | 0 | 6/6 | 21.40 ± 1.74 | 24.456 ± 1.46 | 2.42 ± 0.22 | — |
| Cyclophosphamide | 30 | 6/6 | 21.56 ± 1.54 | 19.16 ± 1.75 | 0.69 ± 0.37*** | 71.29 |
| Isovaleryl | 25 | 6/6 | 21.75 ± 1.23 | 24.41 ± 1.32 | 1.84 ± 0.25** | 23.88 |
| spiramycin I | 50 | 6/6 | 21.53 ± 1.43 | 25.77 ± 1.10 | 1.03 ± 0.44*** | 57.28 |
| | 100 | 6/6 | 21.76 ± 1.08 | 24.12 ± 1.20 | 0.79 ± 0.43*** | 67.49 |
| Isovaleryl | 25 | 6/6 | 20.54 ± 1.45 | 21.16 ± 1.37 | 1.91 ± 0.33* | 20.91 |
| spiramycin II | 50 | 6/6 | 20.07 ± 1.13 | 12.45 ± 1.63 | 1.65 ± 0.32** | 31.61 |
| | 100 | 6/6 | 21.78 ± 1.53 | 12.11 ± 1.20 | 0.92 ± 0.42*** | 62.04 |
| Isovaleryl | 25 | 6/6 | 21.13 ± 1.48 | 23.49 ± 1.02 | 1.47 ± 0.44** | 39.06 |
| spiramycin III | 50 | 6/6 | 21.43 ± 1.85 | 24.06 ± 1.71 | 0.96 ± 0.43*** | 60.25 |
| | 100 | 6/6 | 20.76 ± 2.13 | 23.72 ± 1.60 | 0.90 ± 0.44*** | 62.94 |

*p < 0.05 compared with the model group,
**p < 0.01 compared with the model group,
***p < 0.001 compared with the model group

TABLE 41
Effect of isovaleryl spiramycin I, II and III on the volume change of transplanted
tumor of human thyroid cancer cell TPC-1 in nude mice (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3) (d 30) | Volume (RTV) | Rate (T/C) |
| Model Group | 0 | 6/6 | 119.28 ± 9.59 | 1948.78 ± 84.91 | 16.46 ± 1.92 | — |
| Cyclophosphamide | 30 | 6/6 | 117.10 ± 4.88 | 724.64 ± 285.14*** | 6.16 ± 2.29*** | 37.40 |
| Isovaleryl | 25 | 6/6 | 115.05 ± 9.93 | 1249.28 ± 337.45** | 10.77 ± 2.47** | 65.45 |
| spiramycin I | 50 | 6/6 | 119.45 ± 4.30 | 847.07 ± 356.87*** | 7.15 ± 3.19*** | 43.43 |
| | 100 | 6/6 | 112.69 ± 11.49 | 490.15 ± 322.14*** | 4.63 ± 3.34*** | 28.11 |
| Isovaleryl | 25 | 6/6 | 119.12 ± 3.42 | 1689.63 ± 210.37* | 14.18 ± 1.70 | 86.15 |
| spiramycin II | 50 | 6/6 | 118.54 ± 6.49 | 1148.57 ± 344.78** | 9.72 ± 3.02** | 59.08 |
| | 100 | 6/6 | 117.30 ± 6.48 | 754.42 ± 320.69*** | 6.36 ± 2.38*** | 38.61 |
| Isovaleryl | 25 | 6/6 | 119.35 ± 3.84 | 1210.76 ± 295.50** | 10.19 ± 2.79** | 61.93 |
| spiramycin III | 50 | 6/6 | 116.75 ± 5.22 | 866.73 ± 306.25*** | 7.41 ± 2.51*** | 45.01 |
| | 100 | 6/6 | 115.43 ± 7.58 | 654.78 ± 338.68*** | 5.72 ± 3.04*** | 34.75 |

*p < 0.05 compared with the model group;
**p < 0.01 compared with the model group,
***p < 0.001 compared with the model group

20. Inhibition of Isovaleryl Spiramycin I, II and III on Human Bladder Cancer in Nude Mice Model

Establishment of a Mouse Solid Tumor Model

T-24 cells in a logarithmic growth phase were taken and subjected to a trypan blue exclusion experiment showing that the cell viability was more than 95%, then the cells were subjected to trypsinization, centrifugation, and supernatant removal. Then cell concentration was adjusted to 1×10⁷/ml with matrigel, then each nude mouse was inoculated subcutaneously with 0.2 ml of cells at its right armpit and recorded as the first day of inoculation. When the tumor grows to be greater than or equal to 100 mm³, the mice were randomly divided into 11 groups with 6 mice in each group: isovaleryl spiramycin I groups with doses of 25, 50 and 100 mg/kg, isovaleryl spiramycin II groups with doses of 25, 50 and 100 mg/kg, and isovaleryl spiramycin III groups with doses of 25, 50 and 100 mg/kg. Each group was continuously administered intragastrically for 30 days with a dose of 20 ml/kg. The mice were sacrificed the next day after drug withdrawal and the indicators were tested. The long diameter and short diameter of the tumor, and the body weight of each mouse were recorded every 3 days from drug administration to nude mouse sacrifice.

Calculation of Tumor Volume and Relative Tumor Proliferation Rate

The body weight of the nude mice and the long diameter (a) and short diameter (b) of the transplanted tumor were measured every 3 days, and the tumor volume (v), relative tumor volume (RTV) and relative tumor proliferation rate (T/C) are calculated respectively according to the following formulas, wherein V=a×b²/2; RTV=V/V₀ (V₀ is the tumor volume before administration, V is the tumor volume before sacrifice), and T/C (%)=treatment group RTV/model control group RTV×100%.

Calculation of Tumor Growth Inhibition Rate

Each mouse was weighed and sacrificed. After the tumor was completely stripped off from the body of the mouse, the non-tumor tissues such as blood stains and fat were removed to weigh the tumor and calculate the tumor growth inhibition rate. The average tumor weight of each group of mice is used as an indicator of efficacy. Tumor growth inhibition rate (%)=(1−the average tumor weight of the treatment group/the average tumor weight of the model group)×100%.

The test results show that compared with the control group, each drug-administered group has a certain degree of inhibition on the tumor growth inhibition rate, tumor volume, relative tumor volume and relative tumor proliferation rate (see Table 42, and Table 43).

The tumor growth inhibition rates of isovaleryl spiramycin I groups with low, medium and high doses are 20.55%, 49.20% and 65.84%, respectively.

The tumor growth inhibition rates of isovaleryl spiramycin II groups with low, medium and high doses are 15.57%, 41.99% and 59.25%, respectively.

The tumor growth inhibition rates of isovaleryl spiramycin III groups with low, medium and high doses are 21.00%, 44.84% and 61.30%, respectively.

The tumor volume and relative tumor volume of isovaleryl spiramycin I, II and III groups with low, medium and high doses are significantly lower than those of the model group (P<0.05).

The relative tumor proliferation rates of isovaleryl spiramycin I groups with low, medium and high doses are 77.78%, 58.92% and 33.95%, respectively.

The relative tumor proliferation rates of isovaleryl spiramycin II groups with low, medium and high doses are 81.39%, 64.89% and 46.70%, respectively.

The relative tumor proliferation rates of isovaleryl spiramycin III groups with low, medium and high doses are 80.34%, 62.35% and 39.39%, respectively.

There are no significant changes in the mouse weight of isovaleryl spiramycin I, II and III groups with low, medium and high doses compared with the model group.

TABLE 42
Effect of isovaleryl spiramycin I, II and III on the inhibition rate of transplanted
tumor of human bladder cancer cell T-24 in nude mice (x̅ ± s)

| | | Number of | Body | Body | | |
|---|---|---|---|---|---|---|
| | Dose | Animals | Weight (g) | Weight (g) | Tumor | Inhibition |
| Group | (mg/kg) | (Start/End) | (d 1) | (d 30) | Weight (g) | Rate (%) |
| Model Group | 0 | 6/6 | 19.69 ± 0.72 | 24.75 ± 2.70 | 1.87 ± 0.21 | |
| Cyclophosphamide | 30 | 6/6 | 19.53 ± 0.61 | 27.28 ± 1.43 | 0.51 ± 0.12*** | 73.04 |
| Isovaleryl | 25 | 6/6 | 19.51 ± 0.58 | 23.89 ± 1.18 | 1.49 ± 0.23* | 20.55 |
| spiramycin I | 50 | 6/6 | 19.60 ± 0.70 | 25.41 ± 0.92 | 0.95 ± 0.38*** | 49.20 |
| | 100 | 6/6 | 19.49 ± 0.54 | 24.6 ± 2.08 | 0.64 ± 0.18*** | 65.84 |
| Isovaleryl | 25 | 6/6 | 19.91 ± 0.28 | 24.81 ± 2.42 | 1.58 ± 0.28 | 15.57 |
| spiramycin II | 50 | 6/6 | 19.74 ± 0.82 | 25.84 ± 0.73 | 1.09 ± 0.42** | 41.99 |
| | 100 | 6/6 | 19.51 ± 0.50 | 25.92 ± 1.35 | 0.76 ± 0.19*** | 59.25 |
| Isovaleryl | 25 | 6/6 | 19.57 ± 0.52 | 25.72 ± 1.53 | 1.48 ± 0.43* | 21.00 |
| spiramycin III | 50 | 6/6 | 19.61 ± 0.44 | 26.47 ± 0.52 | 1.03 ± 0.42** | 44.84 |
| | 100 | 6/6 | 19.90 ± 0.58 | 25.05 ± 0.93 | 0.73 ± 0.20*** | 61.30 |

*p < 0.05 compared with the model group,
**p < 0.01 compared with the model group,
***p < 0.001 compared with the model group

TABLE 43
Effect of isovaleryl spiramycin I, II and III on the volume change of transplanted
tumor of human bladder cancer cell T-24 in nude mice (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3) (d 30) | Volume (RTV) | Rate (T/C) |
| Model Group | 0 | 6/6 | 134.41 ± 15.31 | 1772.51 ± 198.69 | 13.21 ± 0.72 | |
| Cyclophosphamide | 30 | 6/6 | 136.87 ± 19.82 | 389.34 ± 82.07 | 2.85 ± 0.50 | 21.60 |
| Isovaleryl | 25 | 6/6 | 139.84 ± 15.80 | 1428.71 ± 156.12 | 10.28 ± 1.12 | 77.78 |
| spiramycin I | 50 | 6/6 | 131.81 ± 7.15 | 1025.16 ± 174.78 | 7.78 ± 1.27 | 58.92 |
| | 100 | 6/6 | 134.21 ± 14.43 | 606.16 ± 136.06 | 4.48 ± 0.75 | 33.95 |
| Isovaleryl | 25 | 6/6 | 132.64 ± 19.75 | 1414.36 ± 188.91 | 10.75 ± 1.34 | 81.39 |
| spiramycin II | 50 | 6/6 | 133.67 ± 5.07 | 1142.42 ± 146.91 | 8.57 ± 1.31 | 64.89 |
| | 100 | 6/6 | 133.57 ± 18.08 | 809.94 ± 131.43 | 6.17 ± 1.26 | 46.70 |
| Isovaleryl | 25 | 6/6 | 135.55 ± 18.08 | 1427.95 ± 311.97 | 10.61 ± 2.24 | 80.34 |
| spiramycin III | 50 | 6/6 | 136.32 ± 15.54 | 1123.28 ± 140.84 | 8.24 ± 0.38 | 62.35 |
| | 100 | 6/6 | 134.53 ± 20.46 | 697.48 ± 130.89 | 5.20 ± 0.71 | 39.39 |

*p < 0.05 compared with the model group;
**p < 0.01 compared with the model group,
***p < 0.001 compared with the model group

21. Inhibition of Transplanted Tumors of Mouse H₂₂ Liver Cancer and Mouse Lewis Lung Cancer

Establishment of a Mouse Solid Tumor Model:

The H₂₂ cell strains cryopreserved in liquid nitrogen were resuscitated in Kunming mice. After 3 generations, the ascites of Kunming mice were taken and placed in a 50 ml centrifuge tube in which 10 ml of 0.9% normal saline was added, and then centrifuging was performed at 1000 rpm for 5 min at room temperature, and the obtained supernatant was removed. Then 10 ml of 0.9% normal saline was added to the tube to blow and mix well, and then the mixture was diluted to 5×10⁶ live cells/ml with normal saline after counting. The tube was stored in ice water, and 75% ethanol was used to disinfect the skin under the right armpits of the mice. Each Kunming mouse was soon inoculated subcutaneously with 0.2 ml of the cells at the armpit of the right forelimb.

Lewis lung cancer cells were cultured in a RPMI 1640 culture medium containing 10% fetal bovine serum at 37° C. in a 5% CO₂ incubator. The cells in the logarithmic growth phase were subjected to trypsinization with 0.25% trypsin, then the cells were collected to be centrifugated to remove the obtained supernatant, then were washed twice with sterile normal saline. And then the cells were suspended in the normal saline to be subjected to a trypan blue staining assay which shows that the cell viability was greater than 95%, and then cell counting was performed. The Lewis cells were adjusted to 1×10⁷/mL in concentration and stored in ice water. 75% ethanol was used to disinfect the skin under the right armpits of the mice, and each C57BL/6 mouse was soon inoculated subcutaneously with 0.2 ml of the cells at its right armpit.

Mouse Grouping and Administration Method

In the H₂₂ liver cancer model, the mice inoculated with the tumor were randomly divided into groups with 10 mice in each group on the next day of inoculation. The groups included: a model control group, a positive drug cyclophosphamide control group (CTX, 26 mg/kg), isovaleryl spiramycin I groups with doses of 13, 26 and 52 mg/kg; isovaleryl spiramycin II groups with doses of 13, 26 and 52 mg/kg; isovaleryl spiramycin III groups with doses of 13, 26 and 52 mg/kg. Each group was continuously administered intragastrically for 7 days with a dose of 20 ml/kg.

In the Lewis lung cancer model, the mice inoculated with the tumor were randomly divided into groups with 10 mice in each group the next day of inoculation. The groups included: a model control group, a positive drug cyclophosphamide control group (CTX, 30 mg/kg), isovaleryl spiramycin I groups with doses of 13, 26 and 52 mg/kg; isovaleryl spiramycin II groups with doses of 13, 26 and 52 mg/kg; isovaleryl spiramycin III groups with doses of 13, 26 and 52 mg/kg. Each group was continuously administered intragastrically for 15 days with a dose of 20 ml/kg.

Calculation of the Tumor Inhibition Rate:

The tumor-bearing mice were weighed and sacrificed the next day after the last administration. The subcutaneous tumors were dissected and weighed. The average tumor weight of each group is calculated, and the tumor inhibition rate is calculated.

Tumor inhibition rate=(1−T/C)×100%

T: average tumor weight of the drug-administered group; C: average tumor weight of the blank control group.

Results:

1. Inhibition of Isovaleryl Spiramycin I, II and III on the Transplanted Tumor of Mouse H₂₂ Liver Cancer

As can be seen from the results of Table 44, the tumor inhibition rate of the positive drug cyclophosphamide control group to Kunming mouse H₂₂ liver cancer is 73.03%. Isovaleryl spiramycin I groups with doses of 13, 26 and 52 mg/kg, isovaleryl spiramycin II groups with doses of 13, 26 and 52 mg/kg, and isovaleryl spiramycin III groups with doses of 13, 26 and 52 mg/kg significantly inhibit the growth of H₂₂ liver cancer in mice.

The positive drug cyclophosphamide group shows a slight decrease in weight compared with the normal control group. The weight of the mice in each of the isovaleryl spiramycin I, II and III groups increases compared with that before the administration, but there is no significant difference compared with the model control group.

TABLE 44
Inhibition of isovaleryl spiramycin I, II and III on the transplanted
tumor of mouse H₂₂ liver cancer (x̅ ± s)

| | | Number of | Body | Body | | |
|---|---|---|---|---|---|---|
| | Dose | Animals | Weight (g) | Weight (g) | | Inhibition |
| Group | (mg/kg) | (Start/End) | (d 1) | (d 7) | Tumor (g) | Rate (%) |
| Model Group | 0 | 10/10 | 23.83 ± 0.43 | 26.13 ± 1.26 | 1.52 ± 0.58 | |
| Cyclophosphamide | 26 | 10/10 | 23.72 ± 1.79 | 23.68 ± 1.32 | 0.41 ± 0.27*** | 73.03 |
| Isovaleryl | 13 | 10/10 | 24.55 ± 1.00 | 26.23 ± 0.79 | 0.88 ± 0.38* | 42.11 |
| spiramycin I | 26 | 10/10 | 23.83 ± 2.36 | 26.68 ± 1.85 | 0.68 ± 0.24** | 55.26 |
| | 52 | 10/10 | 24.02 ± 2.83 | 27.87 ± 1.57 | 0.52 ± 0.68*** | 65.79 |
| Isovaleryl | 13 | 10/10 | 24.22 ± 2.15 | 27.27 ± 2.20 | 1.22 ± 0.31 | 19.74 |
| spiramycin II | 26 | 10/10 | 24.72 ± 1.69 | 27.35 ± 0.80 | 1.06 ± 0.33* | 30.26 |
| | 52 | 10/10 | 23.45 ± 1.69 | 27.02 ± 0.90 | 0.83 ± 0.46** | 45.39 |
| Isovaleryl | 13 | 10/10 | 23.67 ± 3.73 | 26.33 ± 1.43 | 1.01 ± 0.22* | 33.55 |
| spiramycin III | 26 | 10/10 | 24.13 ± 1.46 | 27.47 ± 1.21 | 0.85 ± 0.34* | 44.08 |
| | 52 | 10/10 | 24.32 ± 1.12 | 27.35 ± 0.80 | 0.73 ± 0.45** | 51.97 |

*p < 0.05 compared with the model group;
**p < 0.01 compared with the model group;
^{##}p < 0.05 compared with the cyclophosphamide group

TABLE 45
Effect of isovaleryl spiramycin I, II and III on the tumor volume of transplanted
tumor of KM mouse H₂₂ liver cancer (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3) (d 30) | Volume (RTV) | Rate (T/C) |
| Model Group | 0 | 6/6 | 118.93 ± 13.02 | 1183.27 ± 297.43 | 9.93 ± 2.45 | |
| Cyclophosphamide | 30 | 6/6 | 119.60 ± 31.11 | 307.34 ± 79.91*** | 2.86 ± 1.44*** | 28.78 |
| Isovaleryl | 12.5 | 6/6 | 120.26 ± 24.42 | 657.57 ± 231.56** | 5.41 ± 1.42** | 54.47 |
| spiramycin I | 25 | 6/6 | 118.32 ± 11.90 | 563.69 ± 463.08** | 4.79 ± 0.64** | 48.27 |
| | 50 | 6/6 | 120.68 ± 13.76 | 359.74 ± 480.81*** | 2.96 ± 0.39*** | 29.78 |
| Isovaleryl | 12.5 | 6/6 | 120.80 ± 18.48 | 931.38 ± 141.51* | 7.83 ± 1.50* | 78.85 |
| spiramycin II | 25 | 6/6 | 118.02 ± 10.35 | 779.30 ± 292.34** | 6.56 ± 2.19** | 66.04 |
| | 50 | 6/6 | 118.18 ± 14.68 | 659.02 ± 138.35** | 5.58 ± 0.95** | 56.20 |
| Isovaleryl | 12.5 | 6/6 | 121.58 ± 13.44 | 824.36 ± 143.90* | 6.92 ± 1.88* | 69.68 |
| spiramycin III | 25 | 6/6 | 118.16 ± 21.72 | 650.32 ± 171.23** | 5.65 ± 1.74** | 56.90 |
| | 50 | 6/6 | 122.15 ± 49.44 | 468.19 ± 110.04*** | 4.31 ± 1.65*** | 43.45 |

*p < 0.05 compared with the model group;
**p < 0.01 compared with the model group,
***p < 0.001 compared with the model group

2. Inhibition of Isovaleryl Spiramycin I, II and III on the Transplanted Tumor of Mouse Lewis Lung Cancer

As can be seen from the results of Table 46, the tumor inhibition rate of the positive drug cyclophosphamide control group to mouse Lewis lung cancer is 76.43%. Isovaleryl spiramycin I groups with doses of 13, 26 and 52 mg/kg, isovaleryl spiramycin II groups with doses of 13, 26 and 52 mg/kg, and isovaleryl spiramycin III groups with doses of 13, 26 and 52 mg/kg significantly inhibit the growth of Lewis lung cancer in mice. The weight of the mice in each of the isovaleryl spiramycin I, II and III groups increases compared with that before the administration, but there is no significant difference compared with the model control group.

TABLE 46
Inhibition of isovaleryl spiramycin I, II and III on the transplanted
tumor of mouse Lewis lung cancer (x̅ ± s)

| | | Number of | Body | Body | | |
|---|---|---|---|---|---|---|
| | Dose | Animals | Weight (g) | Weight (g) | Tumor | Inhibition |
| Group | (mg/kg) | (Start/End) | (d 1) | (d 15) | Weight (g) | Rate (%) |
| Model Group | 0 | 10/10 | 19.60 ± 1.07 | 22.70 ± 1.28 | 1.60 ± 0.56 | |
| Cyclophosphamide | 30 | 10/10 | 19.62 ± 1.01 | 21.38 ± 0.67 | 0.38 ± 0.07*** | 76.43 |
| Isovaleryl | 13 | 10/10 | 19.93 ± 1.20 | 22.70 ± 0.87 | 0.85 ± 0.24* | 46.94 |
| spiramycin I | 26 | 10/10 | 19.45 ± 0.68 | 23.43 ± 1.41 | 0.59 ± 0.06** | 62.97 |
| | 52 | 10/10 | 19.87 ± 1.17 | 23.25 ± 0.43 | 0.45 ± 0.14*** | 71.83 |
| Isovaleryl | 13 | 10/10 | 20.33 ± 0.64 | 22.17 ± 1.74 | 0.98 ± 0.37 | 38.67 |
| spiramycin II | 26 | 10/10 | 19.62 ± 1.43 | 22.38 ± 0.96 | 0.89 ± 0.18* | 44.26 |
| | 52 | 10/10 | 20.05 ± 0.67 | 22.75 ± 0.95 | 0.70 ± 0.21** | 56.34 |
| Isovaleryl | 13 | 10/10 | 19.62 ± 1.30 | 22.52 ± 0.57 | 1.03 ± 0.18* | 35.46 |
| spiramycin III | 26 | 10/10 | 19.58 ± 0.63 | 22.28 ± 0.66 | 0.82 ± 0.34** | 48.96 |
| | 52 | 10/10 | 20.00 ± 0.60 | 22.42 ± 1.66 | 0.68 ± 0.18** | 57.54 |

*p < 0.05 compared with the model group;
**p < 0.01 compared with the model group;
^{##}p < 0.05 compared with the cyclophosphamide group

TABLE 47
Effect of isovaleryl spiramycin I, II and III on the tumor volume of transplanted
tumor of KM mouse H₂₂ cells (x̅ ± s)

| | | Number of | | | | Relative Tumor |
|---|---|---|---|---|---|---|
| | Dose | Animals | Tumor Volume | Tumor Volume | Relative Tumor | Proliferation |
| Group | (mg/kg) | (Start/End) | (mm3) (d 1) | (mm3) (d 30) | Volume (RTV) | Rate (T/C) |
| Model Group | 0 | 6/6 | 104.31 ± 16.13 | 1818.28 ± 397.72 | 16.01 ± 4.15 | |
| Cyclophosphamide | 30 | 6/6 | 104.35 ± 30.89 | 371.39 ± 112.68*** | 3.93 ± 1.86*** | 24.55 |
| Isovaleryl | 12.5 | 6/6 | 104.68 ± 20.89 | 955.73 ± 330.24** | 9.22 ± 2.95** | 57.56 |
| spiramycin I | 25 | 6/6 | 104.31 ± 19.76 | 756.20 ± 145.84** | 7.29 ± 0.82** | 45.51 |
| | 50 | 6/6 | 103.79 ± 19.55 | 465.26 ± 52.94*** | 4.65 ± 1.27*** | 29.05 |
| Isovaleryl | 12.5 | 6/6 | 104.18 ± 19.49 | 1304.48 ± 379.51* | 12.89 ± 4.69* | 80.51 |
| spiramycin II | 25 | 6/6 | 103.63 ± 19.46 | 1119.26 ± 338.67* | 11.04 ± 4.07* | 68.97 |
| | 50 | 6/6 | 103.38 ± 18.03 | 798.42 ± 189.19** | 7.81 ± 1.76** | 48.76 |
| Isovaleryl | 12.5 | 6/6 | 105.02 ± 31.31 | 1270.87 ± 198.05* | 13.54 ± 6.73* | 84.57 |
| spiramycin III | 25 | 6/6 | 103.29 ± 29.09 | 841.92 ± 385.16** | 9.35 ± 5.89** | 58.38 |
| | 50 | 6/6 | 104.43 ± 23.09 | 677.35 ± 237.77** | 6.69 ± 2.56** | 41.81 |

*p < 0.05 compared with the model group;
**p < 0.01 compared with the model group,
***p < 0.001 compared with the model group

22. Effect of Isovaleryl Spiramycin I, II and III on Immune Function of Tumor-Bearing Mice

Method

1. Effect on Thymus Index and Spleen Index of Tumor-Bearing Mice

After the tumor-bearing mice are sacrificed, the spleen and thymus are weighed, and the spleen index and thymus index are calculated.

2. Effects on Lymphocyte Proliferation Activity and Natural Killer (NK) Cell Activity of Tumor-Bearing Mice

2.1 Preparation of Spleen Lymphocytes

The serum-free RPMI 1640 medium was placed in a dish, and then the dish was placed on ice. The spleen was aseptically taken and gently ground with a sterile glass slide to prepare a single cell suspension. The single cell suspension was filtered with a double-layer sterile gauze, washed twice with serum-free RPMI1640 medium, and centrifuged at 1,500 rpm for 5 min to remove the obtained supernatant. 2 mL of red blood cell lysate was added to the treated suspension, the mixture was allowed to stand for 2 min, and then 8 mL of RPMI 1640 medium was added, centrifuging was performed at 1,500 rpm for 5 min to remove the obtained supernatant, and then washing was performed twice with the RPMI 1640 medium. Trypan blue staining was performed to count the number of live cells, and the cell viability was more than 95%. A single cell suspension was prepared by using a RPMI 1640 medium containing 10% fetal bovine serum.

2.2 Spleen Lymphocyte Proliferation Activity Assay

The spleen cell suspension was taken, and the cell density was adjusted to 1×10⁷/mL. Each mouse was set with: A. a control well: 100 μL of RPMI 1640 medium was added; B. a concanavalin A (ConA) stimulation well: 100 μL (10 mg/L) of concanavalin A (ConA) solution was added; and C. a bacterial endotoxin (LPS) stimulating well: 100 μL (20 mg/L) of bacterial endotoxin (LPS) solution was added. The above cells were added to a 96-well plate, and then 100 μL of spleen cell suspension was added to each of the above wells. After the culture plate was transferred to a saturated humidity condition with a volume fraction of 5% CO₂ at 37° C. for incubation for 72 h, 10 μL of MTT solution (5 g/L) was added to each well, and incubation was continued to be performed for 4 hours under the same conditions, then the culture was terminated. 100 μl of a triple solution (SDS 10 g, 10M HCl 0.1 mL, was obutanol 5 mL, diluted with distilled water to 100 mL) was added, and the plate was shaken for 10 min to fully dissolve the crystals. The Optical Density (OD) of each well was measured at 570 nm, and the lymphocyte proliferation rate was calculated. Lymphocyte proliferation rate (%)=[(T-C)/C]×100%, wherein T was the Optical Density of the stimulation well, and C is the Optical Density of the control well.

2.3 Natural Killer (NK) Cell Activity Assay

The spleen cell suspension was taken, and the cell density was adjusted to 1×10⁷/mL (effector cells). A suspension of K562 cells was prepared with a cell density of 1×10⁵/mL (target cells). Each mouse was set with: A. effector cells:target cell well (quantity ratio 20:1) to which 20 μL of spleen cell suspension and 100 μL of K562 cell suspension were added; B. an effector cell control well to which 100 μL of spleen cell suspension and 100 μL of RPMI 1640 medium were added; and C. a target cell control well to which 100 μL of K562 cell suspension and 100 μL of RPMI 1640 medium were added. The above cells were added to a 96-well plate. After the 96-well plate was transferred to a saturated humidity condition with a volume fraction of 5% CO₂ at 37° C. for incubation for 22 h, 10 μL of MTT solution (5 g/L) was added to each well, and incubation was continued to be performed for 4 hours under the same conditions, then the culture was terminated. 100 μl of a triple solution (SDS 10 g, 10M HCl 0.1 mL, isobutanol 5 mL, diluted with distilled water to 100 mL) was added, and the plate was shaken for 10 min to fully dissolve the crystals, and the Optical Density (OD) of each well at 490 nm was measured, and the NK cell activity was calculated. NK cell activity (%)=[TO−(S−E)]/TO×100%, wherein TO is the Optical Density of the target cell control well, S is the Optical Density of the effector cell control well, and E is the Optical Density of the effector cell.

Results:

1. Effect on Thymus Index and Spleen Index of H₂₂ Liver Cancer Tumor-Bearing Mice

As can be seen from the results of Table 48, the thymus index and spleen index of the positive drug cyclophosphamide control group are significantly lower than those of the control group (P<0.01). The thymus indexes of the mice in the isovaleryl spiramycin I groups with doses of 13, 26 and 52 mg/kg, isovaleryl spiramycin II groups with doses of 13, 26 and 52 mg/kg, and isovaleryl spiramycin III groups with doses of 13, 26 and 52 mg/kg have no significant change compared with that of the control group.

TABLE 48
Effect of isovaleryl spiramycin I, II and III on thymus index and spleen
index (w) of H₂₂ liver tumor-bearing mice (x̅ ± s)

| | Dose | Spleen Index | Thymus Index |
|---|---|---|---|
| Group | (mg/kg) | (%) | (%) |
| Control Group | 0 | 0.66 ± 0.12 | 0.13 ± 0.05 |
| Cyclophosphamide | 26 | 0.38 ± 0.09** | 0.04 ± 0.02** |
| Isovaleryl | 13 | 0.72 ± 0.07 | 0.15 ± 0.07 |
| spiramycin I | 26 | 0.60 ± 0.15 | 0.14 ± 0.01 |
| | 52 | 0.86 ± 0.25* | 0.12 ± 0.04 |
| Isovaleryl | 13 | 0.59 ± 0.24 | 0.12 ± 0.05 |
| spiramycin II | 26 | 0.68 ± 0.24 | 0.16 ± 0.03 |
| | 52 | 0.64 ± 0.17 | 0.17 ± 0.10 |
| Isovaleryl | 13 | 0.68 ± 0.15 | 0.15 ± 0.06 |
| spiramycin III | 26 | 0.66 ± 0.21 | 0.15 ± 0.05 |
| | 52 | 0.65 ± 0.26 | 0.13 ± 0.02 |

*p < 0.05 compared with the control group;
**p < 0.01 compared with the control group

2. Effect on Thymus Index and Spleen Index of Lewis Lung Cancer Tumor-Bearing Mice

As can be seen from the results in Table 49, the spleen index of the positive drug cyclophosphamide control group is significantly lower than that of the control group (P<0.01). The spleen index and thymus index of the mice in the isovaleryl spiramycin I groups with doses of 13, 26 and 52 mg/kg, isovaleryl spiramycin II groups with doses of 13, 26 and 52 mg/kg, and isovaleryl spiramycin III groups with doses of 13, 26 and 52 mg/kg are not significantly different from those in the control group.

TABLE 49
Effect of isovaleryl spiramycin I, II and III on thymus index and spleen
index (w) in Lewis lung cancer-bearing mice (x̅ ± s, n = 6)

| | Dose | Spleen Index | Thymus Index |
|---|---|---|---|
| Group | (mg/kg) | (%) | (%) |
| Control Group | 0 | 0.76 ± 0.12 | 0.12 ± 0.05 |
| Cyclophosphamide | 26 | 0.48 ± 0.09** | 0.06 ± 0.02** |
| Isovaleryl | 13 | 0.72 ± 0.21 | 0.12 ± 0.03 |
| spiramycin I | 26 | 0.62 ± 0.15 | 0.13 ± 0.04 |
| | 52 | 0.77 ± 0.14* | 0.12 ± 0.06 |
| Isovaleryl | 13 | 0.75 ± 0.24 | 0.12 ± 0.08 |
| spiramycin II | 26 | 0.68 ± 0.21 | 0.14 ± 0.02 |
| | 52 | 0.66 ± 0.17 | 0.15 ± 0.09 |
| Isovaleryl | 13 | 0.62 ± 0.25 | 0.12 ± 0.10 |
| spiramycin III | 26 | 0.68 ± 0.11 | 0.11 ± 0.07 |
| | 52 | 0.74 ± 0.23 | 0.13 ± 0.06 |

**p < 0.01 compared with the control group;
*p < 0.05 compared with the control group;

3. Effect on NK Cell Activity of Lewis Lung Cancer Tumor-Bearing Mice

As can be seen from the results in Table 50, the NK cell activity of the positive drug cyclophosphamide control group is significantly lower than that of the control group (P<0.05). The NK cell activities in isovaleryl spiramycin I groups with doses of 13, 26 and 52 mg/kg, isovaleryl spiramycin II groups with doses of 13, 26 and 52 mg/kg, and isovaleryl spiramycin III groups with doses of 13, 26 and 52 mg/kg significantly increase compared with that of the control group (P<0.01).

TABLE 50
Effect of isovaleryl spiramycin I, II and III on
the NK cell activity of Lewis lung cancer tumor-
bearing (x̅ ± s, n = 6)

| | Dose | NK Cell Activity |
|---|---|---|
| Group | (mg/kg) | (%) |
| Control Group | 0 | 46.2 ± 5.2 |
| Cyclophosphamide | 26 | 35.4 ± 6.6* |
| Isovaleryl | 13 | 52.5 ± 9.2 |
| spiramycin I | 26 | 67.0 ± 12.1** |
| | 52 | 39.8 ± 6.8 |
| Isovaleryl | 13 | 47.2 ± 9.2 |
| spiramycin II | 26 | 47.0 ± 5.5 |
| | 52 | 43.1 ± 7.3 |
| Isovaleryl | 13 | 48.9 ± 6.6 |
| spiramycin III | 26 | 46.0 ± 11.8 |
| | 52 | 49.8 ± 6.2 |

**p < 0.01 compared with the control group;
*p < 0.05 compared with the control group;

4. Effect on Lymphocyte Proliferation Activity of Lewis Lung Cancer Tumor-Bearing Mice

As can be seen from the results in Table 51, the lymphocyte activity of the positive drug cyclophosphamide control group is significantly inhibited (P<0.05). The lymphocyte activities of isovaleryl spiramycin I groups with doses of 13, 26 and 52 mg/kg, isovaleryl spiramycin II groups with doses of 13, 26 and 52 mg/kg, and isovaleryl spiramycin III groups with doses of 13, 26 and 52 mg/kg significantly increase compared with that of the control group (P<0.05, P<0.01).

TABLE 51
Effect of isovaleryl spiramycin I, II and III on lymphocyte proliferation
in transplanted tumor of Lewis lung cancer mice (x̅ ± s, n = 6)

| | | B Lymphocyte | T Lymphocyte |
|---|---|---|---|
| | Dose | Proliferation | Proliferation |
| Group | (mg/kg) | Activity (%) | Activity (%) |
| Control Group | 0 | 30.37 ± 10.16 | 17.60 ± 7.39 |
| Cyclophosphamide | 26 | 11.63 ± 4.68* | 13.24 ± 3.72* |
| Isovaleryl | 13 | 41.63 ± 7.06* | 25.27 ± 8.20** |
| spiramycin I | 26 | 44.81 ± 4.41* | 36.24 ± 2.15** |
| | 52 | 32.71 ± 1.84 | 22.26 ± 4.33 |
| Isovaleryl | 13 | 39.88 ± 3.57 | 16.87 ± 3.28 |
| spiramycin II | 26 | 32.68 ± 2.68 | 15.82 ± 1.68 |
| | 52 | 35.94 ± 3.80 | 19.28 ± 2.35 |
| Isovaleryl | 13 | 33.91 ± 2.65 | 20.17 ± 5.18 |
| spiramycin III | 26 | 34.69 ± 0.34 | 18.51 ± 2.60 |
| | 52 | 32.28 ± 1.27 | 18.25 ± 1.89 |

*p < 0.05 compared with the control group;
**p < 0.01 compared with the control group;

5. Effect on Spleen Index of A549 Lung Cancer Tumor-Bearing Mice

As can be seen from the results in Table 52, the spleen index of the positive drug cyclophosphamide control group is significantly lower than that of the control group (P<0.01). The spleen index of the mice in the isovaleryl spiramycin I groups with doses of 13, 26 and 52 mg/kg, isovaleryl spiramycin II groups with doses of 13, 26 and 52 mg/kg, and isovaleryl spiramycin III groups with doses of 13, 26 and 52 mg/kg do not change significantly compared with that of the control group.

TABLE 52
Effect of isovaleryl spiramycin I, II and III on spleen index of A549 lung
cancer tumor-bearing mice (x̅ ± s, n = 6)

| | Dose | Spleen Index |
|---|---|---|
| Group | (mg/kg) | (%) |
| Control Group | 0 | 0.31 ± 0.04 |
| Cyclophosphamide | 26 | 0.21 ± 0.07* |
| Isovaleryl | 13 | 0.32 ± 0.12 |
| spiramycin I | 26 | 0.38 ± 0.09 |
| | 52 | 0.31 ± 0.08 |
| Isovaleryl | 13 | 0.35 ± 0.06 |
| spiramycin II | 26 | 0.32 ± 0.09 |
| | 52 | 0.37 ± 0.06 |
| Isovaleryl | 13 | 0.32 ± 0.02 |
| spiramycin III | 26 | 0.33 ± 0.11 |
| | 52 | 0.35 ± 0.09 |

*p < 0.05 compared with the control group

The above are only preferred embodiments of the present disclosure, and are not intended to limit the present disclosure in any form. Although the present disclosure has been disclosed in the above preferred embodiments, they are not intended to limit the present disclosure. Any technician who is familiar with the present disclosure can make a slight change or modification into the equivalent embodiments of equivalent changes by using the technical content of the above-mentioned hints without departing from the scope of the technical solution of the present disclosure. But as long as the technical content is not deviated from the technical solution of the present disclosure, any simple modifications, equivalent changes and modifications made to the above embodiments according to the technical substance of the present disclosure are still within the scope of the present disclosure

1. A method for treating or preventing tumor, comprising administering one or more of isovaleryl spiramycin I, II and III to a subject.

2. The method according to claim 1, wherein the tumor includes solid tumor and non-solid tumor.

3. The method according to claim 2, wherein the solid tumor includes benign solid tumor and malignant solid tumor; and the non-solid tumor is lymphoma or leukemia.

4. The method according to claim 3, wherein the malignant solid tumor is breast cancer, liver cancer, lung cancer, renal cancer, brain tumor, cervical cancer, prostate cancer, lymphoma, pancreatic cancer, esophageal cancer, gastric cancer, colon cancer, thyroid cancer, bladder cancer, or malignant skin tumor.

5. The method according to claim 1, comprising administering various formulations made of including one or more of isovaleryl spiramycin I, II and III and pharmaceutically acceptable adjuvants to a subject, or
administering various formulations including pharmaceutically acceptable salts of one or more of isovaleryl spiramycin I, II and III and pharmaceutically acceptable adjuvants to a subject.

6. The method according to claim 1, comprising administering various formulations made of isovaleryl spiramycin I, II and/or III and the pharmaceutically acceptable adjuvants to a subject, or
administering various formulations including the pharmaceutically acceptable salts of one or more of isovaleryl spiramycin I, II and III, anti-tumor drugs and anti-tumor drugs and the pharmaceutically acceptable adjuvants to a subject.

7. The method according to claim 1, comprising administering a combination of a first agent and a second agent to a subject, the first agent contains one or more of isovaleryl spiramycin I, II and III and the pharmaceutically acceptable salts of one or more of isovaleryl spiramycin I, II and III, and the second agent contains an anti-tumor drug.

8. The method according to claim 6, wherein the anti-tumor drug is one or more than one drug selected from a group containing a chemotherapy drug, a radiotherapy drug, a targeted therapy drug, and an immunotherapeutic drug.

9. A medicament for treating and/or preventing tumor, comprising a first active component of the medicament comprises at least one of isovaleryl spiramycin I, II and III.

10. The medicament according to claim 9, wherein the medicament includes a second active component.

11. The medicament according to claim 10, wherein the second active component includes one or more than one drug selected from a group containing a chemotherapy drug, a radiotherapy drug, a targeted therapy drug and an immunotherapeutic drug.

12. A composition for treating and/or preventing tumor, comprising a first agent and a second agent, and an active component of the first agent is at least one of isovaleryl spiramycin I, II and III, wherein,
the second agent includes one or more than one drug selected from a group containing a chemotherapy drug, a radiotherapy drug, a targeted therapy drug and an immunotherapeutic drug.

13. The medicament according to claim 9, wherein the medicament is a pharmaceutically acceptable formulation.

14. The medicament according to claim 13, wherein a dose of isovaleryl spiramycin I, II and/or III in the medicament is in a range from 5 to 1,500 mg.

15. The method according to claim 4, wherein the brain tumor includes glioma or meningioma, and the gastric cancer includes gastric adenocarcinoma.

16. The medicament according to claim 14, wherein the dose of isovaleryl spiramycin I, II and/or III in the medicament is in a range from 50 to 1,000 mg.

17. The medicament according to claim 16, wherein the dose of isovaleryl spiramycin I, II and/or III in the medicament is in a range from 100 to 400 mg.

18. The composition according to claim 12, wherein the first agent is a pharmaceutically acceptable formulation.
